(19) United States
(12) Patent Application Publication
(10) Pub. No.: US 2022/0334108 A1
(43) Pub. Date: Oct. 20, 2022

(54) COMPOSITIONS AND METHODS OF DETECTING ANALYTES

(71) Applicant: 3M INNOVATIVE PROPERTIES COMPANY, St. Paul, MN (US)

(72) Inventors: Frederica Sgolastra, Woodbury, MN (US); Stephen B. Roscoe, Woodbury, MN (US); Daniel M. Dohmeier, White Bear Lake, MN (US); Masayuki Nakamura, Woodbury, MN (US); Catherine A. Bothof, Stillwater, MN (US); Deniz Yuksel Yurt, Woodbury, MN (US); David D. Johnson, Woodbury, MN (US); Kyle A. Kalstabakken, Apple Valley, MN (US); Minghua Dai, Plymouth, MN (US); Myungchan Kang, Woodbury, MN (US); Neil Percy, St. Paul, MN (US); Leslie M. Horton, Hugo, MN (US); Stephen M. Kennedy, St. Paul, MN (US)

(21) Appl. No.: 17/762,945

(22) PCT Filed: Sep. 24, 2020
(86) PCT No.: PCT/IB2020/058951

§ 371 Date: Mar. 23, 2022

Related U.S. Application Data
(60) Provisional application No. 62/906,135, filed on Sep. 26, 2019.

(51) Int. Cl. G01N 33/53; G01N 33/543; G01N 33/552; G01N 33/74; C03C 11/00; C03C 17/30

(52) U.S. Cl. CPC G01N 33/5308; G01N 33/54353; G01N 33/552; G01N 33/743; C03C 11/002; C03C 17/30; G01N 2333/38; C03C 2218/111

(57) ABSTRACT

A composition and method of detecting an analyte in a sample using the composition, the composition including: a liquid that includes water; a plurality of first hollow glass bubbles in the liquid; a plurality of covalently attached first affinity groups that are covalently attached to at least some of the plurality of first hollow glass bubbles; and a plurality of first detector compound molecules not covalently bonded to the plurality of first hollow glass bubbles; wherein the first detector compound molecules include a first detectable group that is detected at a first wavelength; and wherein the first hollow glass bubbles have: a density of less than 0.60 gram/mole; a span of less than 1.0; and a plurality of covalently attached first affinity groups.

### BACKGROUND

Wang et al. (Analytica Chimca Acta 912 (2016) 10-23) disclose nanoparticle-based immunosensors and immunoassays for aflatoxins. Among the sensors disclosed are fluorescence assays, and chemiluminescence assays, and light-scattering assays. Becheva et al. (International Journal of Food Science and Technology 2018) disclose a rapid immunofluorescence assay for staphylococcal enterotoxin A using magnetic nanoparticles. The assay uses antibody immobilized on magnetic nanoparticles. During the assay, magnetic nanoparticles are removed from the liquid by way of a magnet and the fluorescent signal of the supernatant is measured.

Lateral flow assays are known, for example, as self-administered pregnancy tests. These assays do not require special analytical equipment. Quantitation of analytes by ELISA (Enzyme Linked Immunosorbent Assay) is known.

### SUMMARY OF THE DISCLOSURE

The present disclosure provides a composition and method of detecting an analyte (which may be a contaminant) using the composition. The present composition and method combine the rapidity and simplicity of lateral flow assays with the accuracy, quantitation, and batching capability of ELISA. The present composition and method also readily provide for the capability to detect more than one type of analyte in the same assay.

In certain embodiments, the present disclosure provides a composition including: a liquid including water; a plurality of first hollow glass bubbles in the liquid; and a plurality of first detector compound molecules not covalently bonded to the plurality of first hollow glass bubbles, the first detector compound molecules including a first detectable group that is detected at a first wavelength; wherein the first hollow glass bubbles have: a density of less than 0.60 gram/mole; a span of less than 1.0; and a plurality of covalently attached first affinity groups that are covalently attached to at least some of the plurality of first hollow glass bubbles.

In certain embodiments, the present disclosure provides a method of detecting an analyte in a sample, the method including: a) providing a composition that includes: a liquid including water; a plurality of first hollow glass bubbles in the liquid having a plurality of covalently attached first affinity groups that are covalently attached to at least some of the plurality of first hollow glass bubbles; and a plurality of first detector compound molecules not covalently bonded to the plurality of first hollow glass bubbles, the first detector compound molecules including a first detectable group that is detected at a first wavelength; b) adding the sample to the composition, the sample containing first free target analyte molecules; c) allowing the first free target analyte molecules to bind to the first affinity groups or the first detector compound molecules; d) allowing the plurality of first hollow glass bubbles to float to an upper concentrated portion of the liquid; and e) measuring an amount of the first detector compound molecules in a second portion of the liquid that is below the upper concentrated portion, without removing the plurality of first hollow glass bubbles from the liquid. In this method, the first hollow glass bubbles have: a density of less than 0.60 gram/mole; a span of less than 1.0; and a plurality of covalently attached first affinity groups.

The phrase “not covalently bonded” means the detector compounds may be dispersed throughout the liquid in free form, or attached to the glass bubbles in a non-covalent manner (e.g., through hydrogen bonding or through complexation with covalently bonded antibodies).

The term “affinity group” refers to a covalently attached group that is capable of specifically binding another molecule.

The terms “specifically binding” and “specific binding” mean that an affinity group complexes with another molecule (i.e., its complementary molecule) with greater affinity than it complexes with other molecules under the specified conditions.

The term “span” is the particle size distribution of a sample of particles, which can be expressed by the following formula: Span=(90P−10P)/50P, wherein: 90P is the size for which 90 percent (%) of the particles in the distribution are smaller (referred to as the 90^{th} percentile size); 10P is the size for which only 10% of the particles in the distribution are smaller (referred to as the 10^{th} percentile size); 50P is the size for which 50 percent of the particles in the distribution are smaller (referred to as the 50^{th} percentile size).

The term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

The term “or” is generally employed in its usual sense including “and/or” unless the content clearly dictates otherwise.

The term “and/or” means one or all of the listed elements or a combination of any two or more of the listed elements.

Herein, “up to” a number (e.g., up to 50) includes the number (e.g., 50).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### BRIEF DESCRIPTION OF THE DRAWINGS

FIG. 1 is a representation of a method of the present disclosure using a bound antibody elution method with detector compound molecules initially complexed with bound antibodies to detect free (i.e., unbound) target analyte molecules by measuring displaced (i.e., eluted) detector compound molecules.

FIG. 2 is a representation of a method of the present disclosure using a bound antibody depletion method with free detector compound molecules and bound antibodies to detect free target analyte molecules by measuring (remaining free) detector compound molecules.

FIG. 3 is a representation of a method of the present disclosure using a bound target elution method with labelled antibodies (detector compound molecules that include a detector group (i.e., the label) bonded with a complementary group (i.e., the antibody)), which are complexed with bound target molecules, to detect free target analytes by measuring labelled antibodies displaced (i.e., eluted) by the initially free target analyte molecules.

FIG. 4 is a representation of a method of the present disclosure using a bound target depletion method with free initially labelled antibodies (detector compound molecules that include a detector group (i.e., the label) bonded with a complementary group (i.e., the antibody)), to detect free target analytes by measuring labelled antibodies complexed with initially free target analyte molecules.

FIGS. 5A and 5B are size distribution charts of glass bubbles before and after fractionation.

FIGS. 6 and 7 are graphs of the percent increase in fluorescence (at two different wavelengths) over a control sample for solutions containing 10 ppb, 5 ppb, or 1 ppb of either cortisol or fumonisin, or mixtures containing 10 ppb, 5 ppb, or 1 ppb of each.

FIG. 8 is a graph of the percent increase in signal over a control sample for 50 ppb of either cortisol, fumonisin, aflatoxin B1, or a mixture of all three at different excitation and emission wavelengths.

FIG. 9 is a graph of the percent increase in signal over a control sample for 5 ppb of either cortisol, fumonisin, aflatoxin B1, or a mixture of all three at different excitation and emission wavelengths.

FIG. 10 is a graph of fluorescence from solutions containing fumonisin in either grain extract or Phosphate Buffered Saline (PBS).

FIG. 11 is a graph of fluorescence intensity in the detection of aflatoxin B1 by the depletion method.

### DETAILED DESCRIPTION OF ILLUSTRATIVE EMBODIMENTS

Magnetic beads or nanoparticles have been used in detection assays for target analytes. A typical prior-art assay using magnetic beads or nanoparticles is a competitive assay where the beads are modified with molecule, such as an antibody, that can bind to the target analyte. An amount, usually a known amount, of target analyte bound to a detection molecule, such as a luminescent dye, is added to an aqueous liquid containing the metal beads or nanoparticles. The assay is then a competitive binding assay where the detector-bound luminescent analyte and the unbound analyte from the sample compete to bind with the magnetic bead or nanoparticle bound antibody, and the luminescence of the liquid is measured. A magnet is then applied to the aqueous liquid to remove the magnetic beads or nanoparticles, along with whatever is bound to them, from the liquid. The fluorescence of the supernatant is then measured and change in luminescence can be related to the concentration of target analyte in the sample.

There are problems with the use of magnetic beads or nanoparticles. First, the effect of the magnet on the aqueous liquid depends on the proximity of the liquid to the magnet. For large sample volumes, which may particularly be required when the target analyte is of low concentration, the magnet may not be in sufficiently close proximity to the entire sample to ensure that all of the magnetic beads or nanoparticles are removed from the liquid and that the supernatant is free of magnetic beads and nanoparticles.

Second, detection of weak fluorescence or of small differences in luminescence intensity, which may be required to accurately detect or quantify target analytes at low concentrations, is best performed with photomultiplier tubes (sometimes known as PMTs) which have higher sensitivity than other light detectors. PMTs are sensitive to magnetic fields and for good performance should not be operated near magnets. Thus, use of PMTs with magnetic beads or nanoparticles is problematic or impossible.

Third, magnetic beads or nanoparticles, and particularly magnetic nanoparticles, tend to absorb some of the luminescence that is emitted by the assay. This can also reduce the sensitivity of the assay, which can be problematic when the target analytes are present in low concentration.

The three problems above all relate to a general problem of how to detect a target analyte that is present in low concentration, that requires a high degree of sensitivity, or both. One type of target analyte to which these problems particularly apply is mycotoxins, such as aflatoxin.

Yet another problem is that current lateral flow assays do not provide for multiplexing, that is, for detection of more than one type of analyte in the same assay.

Solutions to one or more of the foregoing problems can be found in the present disclosure, which provides a composition and method of detecting an analyte (i.e., target analyte) using the composition. In preferred embodiments, the present composition and method also allow for multiplexing, which is simultaneously measuring levels of more than one analyte at once.

In certain embodiments, the present disclosure provides a composition including: a liquid comprising water; a plurality of first hollow glass bubbles with a plurality of covalently attached first affinity groups in the liquid; and a plurality of first detector compound molecules not covalently bonded to the plurality of first hollow glass bubbles, the first detector compound molecules including a first detectable group that is detected at a first wavelength; wherein the first hollow glass bubbles have: a density of less than 0.60 gram/mole; a span of less than 1.0; and a plurality of covalently attached first affinity groups.

In this context, the phrase “not covalently bonded” means the detector compounds may be dispersed throughout the liquid in free form, or attached to the glass bubbles in a non-covalent manner (e.g., through hydrogen bonding or through complexation with covalently bonded antibodies).

The term “affinity group” refers to a covalently attached group that is capable of specifically binding another molecule. The affinity group may be further complexed with, or bonded to, a detector compound, that can be detected, e.g., by fluorescence. Herein, the affinity group is distinct from the detector compound/group.

In one embodiment, the affinity group is an antibody. Herein, “antibody” includes antibody fragments, and are defined as polypeptide molecules that contain regions that can bind target analytes. Appropriate antibodies can be selected by one of skill in the art. Examples include rabbit anti-mouse IgG antibody, anti-cortisol antibodies, anti-fumonisin antibodies, and anti-aflatoxin B1 antibodies. Suitable antibodies can be obtained from a variety of sources, including Jackson ImmunoResearch, West Grove, Pa., Abcam, Cambridge, Mass., Lifespan Biosciences, Seattle, Wash., and Creative Diagnostics, Shirley, N.Y.

In another embodiment, the affinity group is an aptamer, i.e., a short polynucleotide.

Examples of aptamers include anti-aflatoxin and anti-ochratoxin aptamers, which are available from Aptagen, Jacobus, Pa.

In yet another embodiment, the affinity group is a target analyte molecule, such as a heavy metal or a small organic molecule. Herein, a small organic molecule is one having a molecular weight of no greater than 5000 grams/mole.

The terms “specifically binding” and “specific binding” mean that an affinity group complexes with another molecule (i.e., its complementary molecule) with greater affinity than it complexes with other molecules under the specified conditions. A target analyte molecule is complementary to a bound antibody. An antibody is complementary to a bound target analyte molecule. In various embodiments of the invention, “specifically binding” may mean that an affinity group complexes with a complementary molecule with at least a 10⁶-fold greater affinity, at least a 10⁷-fold greater affinity, at least a 10⁸-fold greater affinity, or at least a 10⁹-fold greater affinity than it complexes with molecules unrelated to the target molecule.

The hollow glass bubbles have a density of less than 0.60 gram/mole. In certain embodiments, the hollow glass bubbles have a density of up to 0.40 gram/mole. In certain embodiments, the hollow glass bubbles have a density of at least 0.05 gram/mole. In certain embodiments, the hollow glass bubbles have a density in a range of 0.05 to 0.40 gram/mole.

In certain embodiments, the hollow glass bubbles have a span of less than 1.0, less than 0.8, or less than 0.7. In certain embodiments, the hollow glass bubbles have a span of at least 0.1, at least 0.2, at least 0.3, at least 0.4, or at least 0.5. In preferred embodiments, the hollow glass bubbles have a span of 0.1 to 1.0, 0.1 to 0.8, 0.1 to 0.7, or 0.1 to 0.5. Typically, the narrower the span, the higher the homogeneity of the hollow glass bubbles, the shorter the assay, and the better the repeatability of the assay.

In certain embodiments, the hollow glass bubbles have an average diameter of at least 30 micrometers, or at least 40 micrometers. In certain embodiments, the hollow glass bubbles have an average diameter of up to 80 micrometers. In certain embodiments, the hollow glass bubbles have an average diameter in a range of 30 to 80 micrometers, or 40 to 80 micrometers. Typically, the larger the hollow glass bubbles, the faster they float to an upper concentrated portion of the liquid for a given density.

Suitable hollow glass bubbles are commercially available from a variety of sources. These include, for example, XLD3000 silica bubbles available from 3M Company, St Paul, Minn. Typically, commercially available hollow glass bubbles are fractionated to obtain the desired size distribution as determined by average diameter (i.e., mean) and span. Such fractionated hollow glass bubbles contribute to the repeatability of the assay.

The advantage of using glass bubbles as a solid support compared to other particle-based assays resides in their inherent buoyancy property that allows for an easy, tunable, and rapid separation from solution upon absence of agitation, without the need of applying magnetic or centrifugal forces.

An additional differentiator of the present methods is that the fluorescence signal is detected in solution rather than on a collected solid phase, as it is most typically done. Hence, this eliminates the requirement for washing steps, which is a significant advantage.

In certain embodiments, the hollow glass bubbles are activated toward affinity group attachment. This can be accomplished, for example, by silanization. Conventional silanization reagents (e.g., 3-glycidoxypropyltrimethoxysilane) and techniques known to one of skill in the art, such as that described in the Examples Section, may be used. Alternatively, glycidyl-functionalized glass bubbles may be used, such as glycidyl-functionalized H20 silica bubbles commercially available from 3M Company, St. Paul, Minn.

In certain embodiments, the composition is multiplexed.

In some of the multiplexed embodiments, the hollow glass bubbles are multiplexed. Such multiplexed hollow glass bubbles include different covalently attached affinity groups. More specifically, such multiplexed hollow glass bubbles include a plurality of covalently attached first affinity groups and a plurality of covalently attached second affinity groups different than the first affinity groups. Or, such multiplexed hollow glass bubbles include a plurality of covalently attached first affinity groups, a plurality of covalently attached second affinity groups different than the first affinity groups, and a plurality of covalently attached third (and optionally fourth, fifth, sixth, seventh, etc.) affinity groups different than the first or second (or any of the other) affinity groups.

In embodiments wherein the multiplexed hollow glass bubbles include two different pluralities of covalently attached affinity groups, the composition includes: a plurality of first detector compound molecules not covalently bonded to the plurality of multiplexed hollow glass bubbles, the first detector compound molecules including a first detectable group that is detected at a first wavelength; and a plurality of second detector compound molecules not covalently bonded to the plurality of multiplexed hollow glass bubbles, the second detector compound molecules including a second detectable group that is detected at a second wavelength that is different than the first wavelength.

In embodiments wherein the multiplexed hollow glass bubbles include three or more different pluralities of covalently attached affinity groups, the composition includes: a plurality of first detector compound molecules not covalently bonded to the plurality of multiplexed hollow glass bubbles, the first detector compound molecules including a first detectable group that is detected at a first wavelength; a plurality of second detector compound molecules not covalently bonded to the plurality of multiplexed hollow glass bubbles, the second detector compound molecules including a second detectable group that is detected at a second wavelength that is different than the first wavelength; and a plurality of third (and optionally fourth, fifth, sixth, seventh, etc.) detector compound molecules not covalently bonded to the plurality of multiplexed hollow glass bubbles, the third (and optionally fourth, fifth, sixth, seventh, etc.) detector compound molecules including a third (and optionally fourth, fifth, sixth, seventh, etc.) detectable group that is detected at a third (and optionally fourth, fifth, sixth, seventh, etc.) wavelength that is different than the first or second wavelength (or any of the other wavelengths).

In some of the multiplexed embodiments, the multiplexed composition includes: a plurality of second hollow glass bubbles in the liquid; a plurality of second affinity groups covalently attached to at least some of the plurality of second hollow glass bubbles; and a plurality of second detector compound molecules not covalently bonded to the plurality of second hollow glass bubbles, the second detector compound molecules including a second detectable group that is detected at a second wavelength that is different than the first wavelength. In such multiplexed compositions, the second hollow glass bubbles are not multiplexed and have: a density of less than 0.60 gram/mole; a span of less than 1.0; and a plurality of covalently attached second affinity groups different than the first affinity groups. Optionally, such compositions may further include multiplexed hollow glass bubbles.

In some of the multiplexed embodiments, the multiplexed composition includes: a plurality of third (and optionally fourth, fifth, sixth, seventh, etc.) hollow glass bubbles in the liquid; and a plurality of third (and optionally fourth, fifth, sixth, seventh, etc.) detector compound molecules not covalently bonded to the plurality of third (and optionally fourth, fifth, sixth, seventh, etc.) hollow glass bubbles, the third (and optionally fourth, fifth, sixth, seventh, etc.) detector compound molecules including a third (and optionally fourth, fifth, sixth, seventh, etc.) detectable group that is detected at a third (and optionally fourth, fifth, sixth, seventh, etc.) wavelength that is different than the first and second (or any of the other) wavelengths. In such multiplexed compositions, the third (and optionally fourth, fifth, sixth, seventh, etc.) hollow glass bubbles are not multiplexed and have: a density of less than 0.60 gram/mole; a span of less than 1.0; and a plurality of covalently attached third (and optionally fourth, fifth, sixth, seventh, etc.) affinity groups different than the first and second (or any of the other) affinity groups. Optionally, such compositions may further include multiplexed hollow glass bubbles.

The present disclosure also provides methods of detecting an analyte (e.g., a contaminant) in a sample. These methods include: a) providing a composition that includes: a liquid including water; a plurality of first hollow glass bubbles in the liquid, the first hollow glass bubbles having a a plurality of covalently attached first affinity groups that are covalently attached to at least some of the plurality of first hollow glass bubbles; and a plurality of first detector compound molecules not covalently bonded to the plurality of first hollow glass bubbles, the first detector compound molecules including a first detectable group that is detected at a first wavelength; b) adding the sample to the composition, the sample containing first free target analyte molecules; c) allowing the first free target analyte molecules to bind to the first affinity groups or the first detector compound molecules; d) allowing the plurality of first hollow glass bubbles to float to an upper concentrated portion of the liquid; and e) measuring an amount of the first detector compound molecules in a second portion of the liquid that is below the upper concentrated portion, without removing the plurality of first hollow glass bubbles from the liquid. In this method, the first hollow glass bubbles have: a density of less than 0.60 gram/mole; a span of less than 1.0; and a plurality of covalently attached first affinity groups. It is understood that not all the bubbles may float to the upper concentrated portion; however, greater sensitivity results from more efficient separation.

The methods described herein may be used for detecting heavy metals and small organic compounds. Such metals and compounds are those typically found contaminating food (e.g., animal feed) and water (i.e., a food or water contaminant), and those used as biomarkers.

Herein, a “heavy metal” is a metal or metalloid having an atomic number of greater than 20 (calcium). Typically, such heavy metal has an atomic number of no greater than 92 (uranium).

Examples include Pb (lead), As (arsenic), and Hg (mercury).

The methods are particularly effective for small organic molecules. Such small organic molecules have a molecular weight of no greater than 5000 grams/mole. Examples of such small organic molecules include: mycotoxins, such as aflatoxins (e.g., B1, B2, G1, G2, and M1), trichothecenes (e.g., deoxynivalenol, HT-2, and T-2), zearalenone, ochratoxins (e.g., A, B, C, and TA), fumonisins (e.g., B1, B2, B3, and B4), and patulin; shellfish toxins, such as saxitoxin, okadaic acid, domoic acid, and dinophysistoxin derivatives; hormones, such as cortisol, progesterone, estradiol, androstenedione, diethylstilbestrol, ractopamine, clenbuterol, stanozolol, triamcinolone, zilpaterol, zeranol, and trenbolone; antibiotics and antivirals, such as beta-lactams, tetracyclines, sulfonamides, nitrofurans, ciprofloxacin, chloramphenicol, acyclovir, triclosan, streptomycin, amphenicol, flunixin, florfenicol, fluoroquinolone, gentamycin, lincomycin, neomycin, tylosin, diclazuril, dimetridazole, colistin, and dapsone; biomarkers, such as coprostanol, cholesterol, and creatinine; pesticides, such as atrazine, N,N-diethyl-meta-toluamide (DEET), glyphosate, polychlorinated biphenyls (e.g., DDT and DDE), bromacil, avermectin, organophosphates, and N-methylcarbamates; drugs of abuse, such as opioids (e.g., fentanyl and heroin), cocaine, tetrahydrocannabinol (THC), and amphetamines; and other bioactive compounds, such as dexamethasone, albuterol, caffeine, warfarin, ibuprofen, codeine, lidocaine, celecoxib, benzatropine, metoprolol, omeprazole, nadolol, statins (e.g., atorvastatin and simvastatin), lisinopril, ketamine, and nandrolone. Such small organic molecules do not include proteins. Particularly important small organic molecules to detect include mycotoxins.

The samples that include such analytes can be a solid or a liquid, but are in the form of a liquid when fluorescence intensity is measured. The samples are typically food (e.g., grains such as oats, corn, wheat) or water samples. If a solid sample is to be analyzed, the target analyte is preferably extracted using aqueous extraction techniques to avoid handling and disposal of organic solvents. Typical aqueous extraction techniques use surfactant-based extraction. For example, TWEEN 20 and TRITON-X nonionic detergents can be used to extract the target analyte. If interference is observed, this may be overcome by dilution in a buffer (e.g., phosphate buffered saline).

As noted above, the affinity group may be further complexed with, or noncovalently bonded to, a detector compound that can be detected, e.g., by fluorescence. Herein, the affinity group is distinct from the detector compound/group.

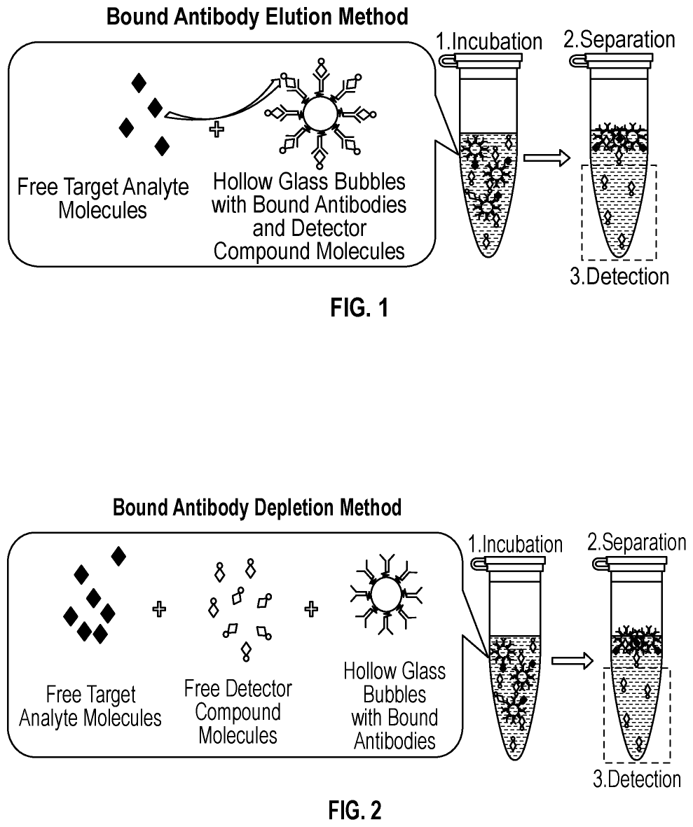

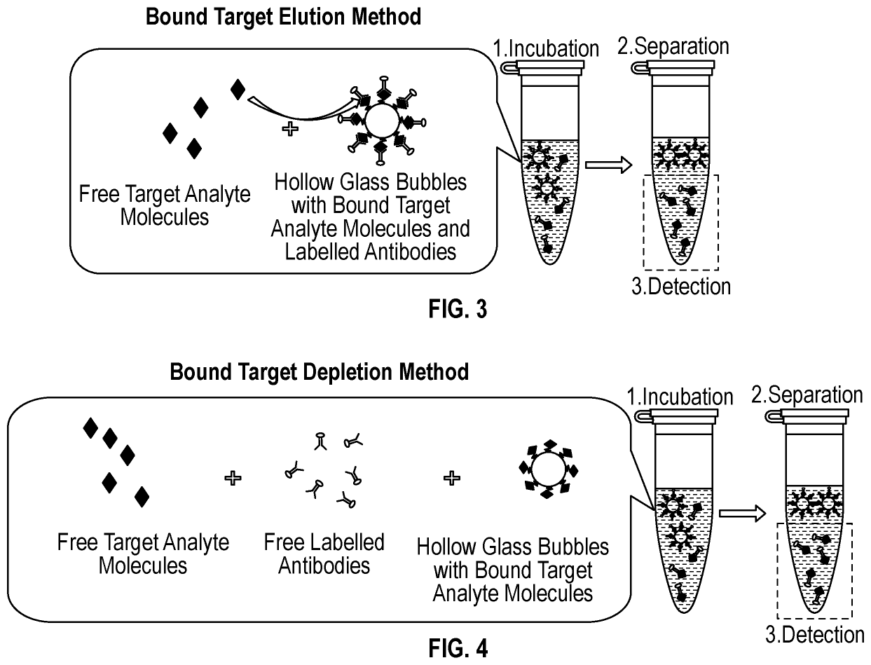

The detector compound molecules include a detector group that can be detected, e.g., by fluorescence, and a complementary group. Such detector compounds (i.e., a detector group bonded to a complementary group) initially are complexed with bound antibodies in the Bound Antibody Elution Method (as shown in FIG. 1). The detector compound molecules are labelled antibodies, which are initially complexed with bound target molecules, in the Bound Target Elution Method (as shown in FIG. 3). Alternatively, the detector compound initially is in the form of free molecules in the Bound Antibody Depletion Method (as shown in FIG. 2). Or, the detector compound initially is in the form of free labelled antibodies in the Bound Target Depletion Method (as shown in FIG. 4).

Such detector compound molecules may include a fluorescent group as the detector group. Thus, the methods described herein may be used for detecting a fluorescent signal, and in certain embodiments, measuring the amount of a fluorescent signal, e.g., a first fluorescent signal at a first wavelength.

For example, the amine-reactive TEXAS RED-X succinimidyl ester can be used to create bright red-fluorescent detector compounds with excitation/emission maxima of approximately 595/615 nanometers (nm). Fluorescein isothiocyanate can be used to provide fluorescence at 520 nm emission, with 488 nm excitation. The amount of fluorescent signal (i.e., fluorescent intensity) is the measurement of the emitted light. A fluorescent signal can be detected using a fluorescence detector, which consists of a light source, an excitation filter or monochromator, an emission filter or monochromator, and a detector, often a photomultiplier tube (PMT).

In certain embodiments, the intensity of the fluorescent signal is directly proportional to the amount of target analyte in a sample. Thus, in such embodiments, the methods described herein can be used to quantify the amount of target analyte. This can be done by comparing the signal of an unknown sample with that of standards (e.g., typically three or more standard samples) of known target analyte concentrations.

The methods may include multiplexed compositions, which may or may not include multiplexed hollow glass bubbles, as described herein, and thereby detect multiple analytes in a sample.

The methods of the present disclosure allow for simplified workflow. For example, in one embodiment, after a standard extraction process, a single pipetting step is all that is required to add sample to a testing tube containing, for example, glass bubbles conjugated with an affinity group, and a detector compound, and after a short period of mixing (e.g., 10 min), tubes can be inserted into a reader to measure fluorescence from the solution. Although certain embodiments may only require a single step, the detection portion of other embodiments may benefit from filtration.

A set of standards analyzed in parallel is used for quantification. More than one sample can be quantified with a single set of standards, thus allowing for batching. This is a significant improvement over cumbersome ELISA workflows, and is comparable to lateral flow protocols. Moreover, the possibility of testing multiple target analytes per tube offers an increased benefit and improves user productivity.

A major problem with fluorescence immunoassays has been interference due to endogenous matrix components. Depending on the detector compound used, this interference can take the form of absorption (“quenching”) of the detector compound's fluorescent output, or high background at the wavelength being detected. The result of this interference by matrix components is a loss in assay sensitivity, thus limiting the use of fluorescence immunoassays to detection of compounds at relatively high concentrations in complex matrices. Using the methods of the present disclosure, the background interference associated with food matrix may be reduced (or eliminated) by moving the detection wavelength to a higher range of the visible spectrum (e.g., greater than 600 nm).

An additional advantage of the present methods is the reduction in handling of potentially toxic target analytes.

### Bound Antibody Compositions/Methods

The bound antibody methods of the present disclosure are competitive binding (e.g., competitive complexation) assays, wherein detector compounds compete with target analytes present in a liquid sample to complex with a limited number of antibody binding sites available on the hollow glass bubbles. After binding, the hollow glass bubbles float to an upper concentrated portion of the liquid, and the fluorescence emitting from the unbound (i.e., free) detector compound molecules in the liquid below that of the upper concentrated portion is measured. It is understood that not all the glass bubbles may float to the upper concentrated portion of the liquid; however, greater sensitivity results from more efficient separation.

In the bound antibody methods, the composition includes: a liquid including water; a plurality of first hollow glass bubbles in the liquid; and a plurality of first detector compound molecules not covalently bonded to the plurality of first hollow glass bubbles, the first detector compounds including a first detectable group that is detected at a first wavelength; wherein the first hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached first affinity groups. Such composition is referred to herein as the bound antibody composition.

This bound antibody composition can be used in either an elution or depletion method. In the elution method, the hollow glass bubbles having antibodies bound thereto are complexed with detector compound molecules before being exposed to the sample. The target analyte in the sample will displace, or elute, the detector compound molecules from the bubbles. In the depletion method, the detector compound molecules are added to a liquid sample containing the target analyte and the two compete for binding to the antibody on the glass bubbles.

In the elution method, as shown in FIG. 1, antibodies are the affinity groups that are covalently attached to the hollow glass bubbles. The antibodies are complexed with the detector compound molecules prior to contact with free target analyte molecules, thereby forming one type of bound antibody composition. The detector compound molecules and the target analyte molecules both include a complementary group allowing them to compete for binding to the bound antibodies. In this method of detecting an analyte, the method includes: providing a bound antibody composition (that includes hollow glass bubbles with first bound antibodies as the affinity groups, which are complexed with first detector compound molecules); adding a sample to the composition, the sample containing first free target analyte molecules; allowing the first free target analyte molecules to bind to the first bound antibodies (thereby displacing or eluting the first detector compound molecules from the first bound antibodies covalently bonded to the hollow glass bubbles); allowing the plurality of first hollow glass bubbles to float to an upper concentrated portion of the liquid; and measuring an amount of the (displaced) first detector compound molecules in a second portion of the liquid that is below the upper concentrated portion, without removing the plurality of first hollow glass bubbles from the liquid.

In the depletion method, as shown in FIG. 2, antibodies are the affinity groups that are covalently attached to the hollow glass bubbles. A competition is set up between target analyte molecules and detector compound molecules for binding to bound antibodies. In the composition for use in this method, the first detector compound molecules include a first complementary group and the first free target analyte molecules include the first complementary group, wherein the first detector compound molecules compete with the first free target analyte molecules for complexation with the first bound antibodies. In this method of detecting an analyte, the method includes: providing a bound antibody composition and free detector compound molecules; adding a sample to the composition, the sample containing first free target analyte molecules; allowing the first free target analyte molecules to bind to the first bound antibodies (thereby competing with the first free detector compound molecules); allowing the plurality of first hollow glass bubbles to float to an upper concentrated portion of the liquid; and measuring an amount of the (remaining free) first detector compound molecules in a second portion of the liquid that is below the upper concentrated portion, without removing the plurality of first hollow glass bubbles from the liquid.

### Bound Target Compositions/Methods

The bound target methods of the present disclosure are competitive binding (e.g., competitive complexation) assays, wherein target analytes present in a liquid sample compete with the bound target analyte molecules on the glass bubbles to complex with a limited number of labelled antibodies. After complexation, the hollow glass bubbles float to an upper concentrated portion of the liquid, thus removing all labelled antibody complexed with bound target analyte molecules on the glass bubbles from solution, and the fluorescence emitting from the labelled antibodies complexed with previously free target analyte molecules of the liquid below that of the upper concentrated portion is measured. It is understood that not all the bubbles may float to the upper concentrated portion; however, greater sensitivity results from more efficient separation.

In the bound target methods, the composition includes: a liquid including water; a plurality of first hollow glass bubbles in the liquid; and a plurality of first labelled antibodies not covalently bonded to the plurality of first hollow glass bubbles, the first labelled antibodies including a first detectable group that is detected at a first wavelength; wherein the first hollow glass bubbles have: a density of less than 0.60 gram/mole; a span of less than 1.0; and a plurality of covalently attached first bound target analyte molecules. In such compositions, the “bound” target analyte molecules may be covalently bonded or otherwise attached to the glass bubbles. Such composition is referred to herein as the bound target composition.

This composition can be used in either an elution or depletion method. In the elution method, the hollow glass bubbles having target analyte molecules bound thereto are complexed with labelled antibodies before being exposed to the sample. The free target analyte molecules in the sample will displace, or elute, the labelled antibodies from the bound target analyte molecules covalently bonded to, or otherwise bonded to, the hollow glass bubbles. In the depletion method, the labelled antibodies are added to a liquid sample containing the free target analyte molecules and the hollow glass bubbles having the bound target analyte molecules attached thereto. The free and bound target analyte molecules compete for complexation with the labelled antibodies.

In the elution method, as shown in FIG. 3, target analyte molecules are bound to the glass bubbles and complexed with the labelled antibodies prior to contact with free target analyte molecules. The target analyte molecules are covalently bonded to, or otherwise bound to, the hollow glass bubbles and complexed with the labelled antibodies, which are thereby attached (but not covalently) to the glass bubbles. In this method of detecting an analyte, the method includes: providing a bound target composition (that includes hollow glass bubbles with bound target analyte molecules as the affinity groups, which are complexed with labelled antibodies); adding a sample to the composition, the sample containing first free target analyte molecules; allowing the first free target analyte molecules to complex with the first labelled antibodies (thereby displacing or eluting the first labelled antibodies from the first bound target analyte molecules attached to the hollow glass bubbles); allowing the plurality of first hollow glass bubbles to float to an upper concentrated portion of the liquid; and measuring an amount of the first detector compound molecules (the displaced first labelled antibodies complexed with initially first free target analyte molecules) in a second portion of the liquid that is below the upper concentrated portion, without removing the plurality of first hollow glass bubbles from the liquid.

In the depletion method, as shown in FIG. 4, a competition is set up between bound target analyte molecules and free target analyte molecules for complexation with labelled antibodies.

The composition for use in this method includes first free labelled antibodies and hollow glass bubbles having first bound target analyte molecules attached thereto. The first bound target analyte molecules compete with the first free target analyte molecules for complexation with the first free labelled antibodies. In this method of detecting an analyte, the method includes: providing a bound target composition (that includes hollow glass bubbles with first bound target analyte molecules as the affinity groups and free labelled antibodies as the first detector compound molecules); adding a sample to the composition, the sample containing first free target analyte molecules; allowing the first free target analyte molecules to complex with the first labelled antibodies (thereby competing with the first bound target analyte molecules); allowing the plurality of first hollow glass bubbles to float to an upper concentrated portion of the liquid; and measuring an amount of the first labelled antibodies complexed with initially free target analyte molecules in a second portion of the liquid that is below the upper concentrated portion, without removing the plurality of first hollow glass bubbles from the liquid.

### EXEMPLARY EMBODIMENTS

Embodiment 1 is a composition comprising: a liquid comprising water; a plurality of first hollow glass bubbles in the liquid; and a plurality of first detector compound molecules not covalently bonded to the plurality of first hollow glass bubbles, the first detector compound molecules comprising a first detectable group that is detected at a first wavelength; wherein the first hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached first affinity groups.

Embodiment 2 is the composition of embodiment 1, wherein the first affinity groups comprise first bound antibodies (as represented by FIGS. 1 and 2).

Embodiment 3 is the composition of embodiment 2, wherein the first bound antibodies are complexed with the first detector compound molecules prior to contact with first free target analyte molecules (as represented by FIG. 1).

Embodiment 4 is the composition of embodiment 2 or 3, wherein the first detector compound molecules comprise a first complementary group and the composition further comprises first free target analyte molecules comprising the first complementary group, wherein the first detector compound molecules compete with the first free target analyte molecules for binding to the first bound antibodies (as represented by FIGS. 1 and 2).

Embodiment 5 is the composition of embodiment 1, wherein the first affinity groups comprise first bound target analyte molecules and the first detector compound molecules comprise first labelled antibodies (as represented by FIGS. 3 and 4).

Embodiment 6 is the composition of embodiment 5, wherein the first affinity groups are complexed with the first labelled antibodies prior to contact with first free target analyte molecules (as represented by FIG. 3).

Embodiment 7 is the composition of embodiment 5 or 6, wherein the composition further comprises first free target analyte molecules, wherein the first bound target analyte molecules compete with the first free target analyte molecules for complexation with the first labelled antibodies (as represented by FIGS. 3 and 4).

Embodiment 8 is the composition of any of the previous embodiments, wherein the first hollow glass bubbles are concentrated in an upper portion of the liquid.

Embodiment 9 is the composition of any of the previous embodiments, wherein the first hollow glass bubbles have a density of at least 0.05 gram/mole.

Embodiment 10 is the composition of any of the previous embodiments, wherein the first hollow glass bubbles have a span of at least 0.1, at least 0.2, at least 0.3, at least 0.4, or at least 0.5.

Embodiment 11 is the composition of any of the previous embodiments, wherein the first hollow glass bubbles have a span of 0.1 to 1.0, 0.1 to 0.8, 0.1 to 0.7, or 0.1 to 0.5.

Embodiment 12 is the composition of any of the previous embodiments, wherein the first hollow glass bubbles have an average diameter in a range of 30 to 80 micrometers, or 40 to 80 micrometers.

Embodiment 13 is the composition of any of the previous embodiments, wherein the composition is multiplexed.

Embodiment 14 is the composition of embodiment 13, wherein the first hollow glass bubbles are multiplexed.

Embodiment 15 is the composition of embodiment 14, wherein: the multiplexed hollow glass bubbles further comprise a plurality of covalently attached second affinity groups different than the first affinity groups; and the composition further comprises a plurality of second detector compound molecules not covalently bonded to the plurality of multiplexed hollow glass bubbles, the second detector compound molecules comprising a second detectable group that is detected at a second wavelength that is different than the first wavelength.

Embodiment 16 is the composition of any of embodiments 13 through 15, wherein the multiplexed composition further comprises:

a plurality of second hollow glass bubbles in the liquid, wherein the second hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached first affinity groups; and a plurality of covalently attached second affinity groups different than the first affinity groups; and

a plurality of second detector compound molecules not covalently bonded to the plurality of second hollow glass bubbles, the second detector compound molecules comprising a second detectable group that is detected at a second wavelength that is different than the first wavelength.

Embodiment 17 is the composition of embodiment 15 or 16, wherein the second affinity groups comprise second bound antibodies.

Embodiment 18 is the composition of embodiment 17, wherein the second bound antibodies are complexed with the second detector compound molecules prior to contact with second free target analyte molecules.

Embodiment 19 is the composition of embodiment 17 or 18, wherein the second detector compound molecules comprise a second complementary group, and the composition further comprises second free target analyte molecules comprising the second complementary group, wherein the second detector compound molecules compete with the second free target analyte molecules for binding to the second affinity groups.

Embodiment 20 is the composition of embodiment 15 or 16, wherein the second affinity groups comprise second bound target analyte molecules and the second detector compound molecules comprise second labelled antibodies.

Embodiment 21 is the composition of embodiment 20, wherein the second affinity groups are complexed with the second labelled antibodies prior to contact with second free target analyte molecules.

Embodiment 22 is the composition of embodiment 20, wherein the composition further comprises second free target analyte molecules, wherein the second bound target analyte molecules compete with the second free target analyte molecules for complexation with the second labelled antibodies.

Embodiment 23 is the composition of any of embodiments 16 through 22, wherein the second hollow glass bubbles are concentrated in an upper portion of the liquid.

Embodiment 24 is the composition of any of embodiments 16 through 23, wherein the second hollow glass bubbles have a density of at least 0.05 gram/mole.

Embodiment 25 is the composition of any of embodiments 16 through 24, wherein the second hollow glass bubbles have a span of at least 0.1, at least 0.2, at least 0.3, at least 0.4, or at least 0.5.

Embodiment 26 is the composition of any of embodiments 16 through 25, wherein the second hollow glass bubbles have a span of 0.1 to 1.0, 0.1 to 0.8, 0.1 to 0.7, or 0.1 to 0.5.

Embodiment 27 is the composition of any of embodiments 16 through 26, wherein the second hollow glass bubbles have an average diameter in a range of 30 to 80 micrometers, or 40 to 80 micrometers.

Embodiment 28 is a method of detecting an analyte in a sample, the method comprising:

a) providing a composition comprising:
a liquid comprising water;
a plurality of first hollow glass bubbles in the liquid, wherein the first hollow glass bubbles have:
   a density of less than 0.60 gram/mole, or up to 0.40 gram/mole;
   a span of less than 1.0, less than 0.8, or less than 0.7;
   optionally, an average diameter of at least 30 micrometers, or at least 40 micrometers; and
   a plurality of covalently attached first affinity groups; and
a plurality of first detector compound molecules not covalently bonded to the plurality of first hollow glass bubbles, the first detector compound molecules comprising a first detectable group that is detected at a first wavelength;

b) adding the sample to the composition, the sample containing first free target analyte molecules;

c) allowing the first free target analyte molecules to bind to the first affinity groups or the first detector compound molecules;

d) allowing the plurality of first hollow glass bubbles to float to an upper concentrated portion of the liquid; and

e) measuring an amount of the first detector compound molecules in a second portion of the liquid that is below the upper concentrated portion, without removing the plurality of first hollow glass bubbles from the liquid.

Embodiment 29 is the method of embodiment 28, wherein the analyte is a food or water contaminant.

Embodiment 30 is the method of embodiment 28 or 29, wherein the analyte is a heavy metal or a small organic molecule.

Embodiment 31 is the method of embodiment 30, wherein the analyte is a small organic molecule.

Embodiment 32 is the method of embodiment 31, wherein the small organic molecule is selected from a mycotoxin, shellfish toxin, hormone, antibiotic, antiviral, biomarker, pesticide, drug of abuse, and other bioactive molecule.

Embodiment 33 is the method of embodiment 31 or 32, wherein the small organic molecule is selected from aflatoxin B1, aflatoxin B2, aflatoxin G1, aflatoxin G2, aflatoxin M1, deoxynivalenol, trichothecene HT-2, trichothecene T-2, zearalenone, ochratoxin A, ochratoxin B, ochratoxin C, ochratoxin TA, fumonisin B1, fumonisin B2, fumonisin B3, fumonisin B4, patulin, saxitoxin, okadaic acid, domoic acid, a dinophysistoxin derivative, cortisol, progesterone, estradiol, androstenedione, diethylstilbestrol, ractopamine, clenbuterol, stanozolol, triamcinolone, zilpaterol, zeranol, trenbolone, a beta-lactam, a tetracycline, a sulfonamide, a nitrofuran, ciprofloxacin, chloramphenicol, acyclovir, triclosan, streptomycin, amphenicol, flunixin, florfenicol, fluoroquinolone, gentamycin, lincomycin, neomycin, tylosin, diclazuril, dimetridazole, colistin, dapsone, coprostanol, cholesterol, creatinine, atrazine, N,N-Diethyl-meta-toluamide (DEET), glyphosate, a polychlorinated biphenyl, bromacil, avermectin, an organophosphates, an N-methylcarbamate; an opioid, cocaine, tetrahydrocannabinol (THC), an amphetamine, dexamethasone, albuterol, caffeine, warfarin, ibuprofen, codeine, lidocaine, celecoxib, benzatropine, metoprolol, omeprazole, nadolol, a statin, lisinopril, ketamine, and nandrolone.

Embodiment 34 is the method of any of embodiments 28 through 33, wherein measuring the amount of the first detector compound molecules is based on measurement of a fluorescent signal at the first wavelength.

Embodiment 35 is the method of any of embodiments 28 through 34, wherein the first affinity groups comprise first bound antibodies (as represented by FIGS. 1 and 2).

Embodiment 36 is the method of embodiment 35, wherein the first bound antibodies are complexed with the first detector compound molecules prior to contact with first free target analyte molecules (as represented by FIG. 1).

Embodiment 37 is the method of embodiment 35 or 36, wherein the first detector compound molecules comprise a first complementary group and the first free target analyte molecules comprise the first complementary group, wherein the first detector compound molecules compete with the first free target analyte molecules for binding to the first bound antibodies (as represented by FIGS. 1 and 2).

Embodiment 38 is the method of any of embodiments 28 through 35, wherein the first affinity groups comprise first bound target analyte molecules and the first detector compound molecules comprise first labelled antibodies (as represented by FIGS. 3 and 4).

Embodiment 39 is the method of embodiment 38, wherein the first affinity groups are complexed with the first labelled antibodies prior to contact with first free target analyte molecules (as represented by FIG. 3).

Embodiment 40 is the method of embodiment 38 or 39, wherein the composition further comprises first free target analyte molecules, wherein the first bound target analyte molecules compete with the first free target analyte molecules for complexation with the first free labelled antibodies (as represented by FIGS. 3 and 4).

Embodiment 41 is the method of any of embodiments 28 through 40, wherein the first hollow glass bubbles are concentrated in an upper portion of the liquid.

Embodiment 42 is the method of any of embodiments 28 through 41, wherein the first hollow glass bubbles have a density of at least 0.05 gram/mole.

Embodiment 43 is the method of any of embodiments 28 through 42, wherein the first hollow glass bubbles have a span of at least 0.1, at least 0.2, at least 0.3, at least 0.4, or at least 0.5.

Embodiment 44 is the method of any of embodiments 28 through 43, wherein the first hollow glass bubbles have a span of 0.1 to 1.0, 0.1 to 0.8, 0.1 to 0.7, or 0.1 to 0.5.

Embodiment 45 is the method of any of embodiments 28 through 44, wherein the first hollow glass bubbles have an average diameter in a range of 30 to 80 micrometers, or 40 to 80 micrometers.

Embodiment 46 is the method of any of embodiments 28 through 45, wherein the composition is multiplexed.

Embodiment 47 is the method of embodiment 46, wherein the first hollow glass bubbles are multiplexed.

Embodiment 48 is the method of embodiment 47, wherein: the multiplexed hollow glass bubbles further comprise a plurality of covalently attached second affinity groups different than the first affinity groups; and the composition further comprises a plurality of second detector compound molecules not covalently bonded to the plurality of multiplexed hollow glass bubbles, the second detector compound molecules comprising a second detectable group that is detected at a second wavelength that is different than the first wavelength.

Embodiment 49 is the method of any of embodiments 46 through 48, wherein the multiplexed composition further comprises:

a plurality of second hollow glass bubbles in the liquid, wherein the second hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached second affinity groups different than the first affinity groups; and a plurality of second detector compound molecules not covalently bonded to the plurality of second hollow glass bubbles, the second detector compound molecules comprising a second detectable group that is detected at a second wavelength that is different than the first wavelength.

Embodiment 50 is the method of embodiment 48 or 49, wherein the second affinity groups comprise second bound antibodies (as represented by FIGS. 1 and 2).

Embodiment 51 is the method of embodiment 50, wherein the second bound antibodies are complexed with the second detector compound molecules prior to contact with second free target analyte molecules (as represented by FIG. 1).

Embodiment 52 is the method of embodiment 50 or 51, wherein the second detector compound molecules comprise a second complementary group, and the composition further comprises second free target analyte molecules comprising the second complementary group, wherein the second detector compound molecules compete with the second free target analyte molecules for binding to the second bound antibodies (as represented by FIGS. 1 and 2).

Embodiment 53 is the method of embodiment 48 or 49, wherein the second affinity groups comprise second bound target analyte molecules and the second detector compound molecules comprise second labelled antibodies (as represented by FIGS. 3 and 4).

Embodiment 54 is the method of embodiment 53, wherein the second affinity groups are complexed with the second labelled antibodies prior to contact with second free target analyte molecules (as represented by FIG. 3).

Embodiment 55 is the method of embodiment 53 or 54, wherein the composition further comprises second free target analyte molecules, wherein the second bound target analyte molecules compete with the second free target analyte molecules for complexation with the second labelled antibodies (as represented by FIGS. 3 and 4).

Embodiment 56 is the method of any of embodiments 49 through 55, wherein the second hollow glass bubbles are concentrated in an upper portion of the liquid.

Embodiment 57 is the method of any of embodiments 49 through 56, wherein the second hollow glass bubbles have a density of at least 0.05 gram/mole.

Embodiment 58 is the method of any of embodiments 49 through 57, wherein the second hollow glass bubbles have a span of at least 0.1, at least 0.2, at least 0.3, at least 0.4, or at least 0.5.

Embodiment 59 is the method of any of embodiments 49 through 58, wherein the second hollow glass bubbles have a span of 0.1 to 1.0, 0.1 to 0.8, 0.1 to 0.7, or 0.1 to 0.5.

Embodiment 60 is the method of any of embodiments 49 through 59, wherein the second hollow glass bubbles have an average diameter in a range of 30 to 80 micrometers, or 40 to 80 micrometers.

Embodiment 61 is a bound antibody composition comprising: a liquid comprising water; a plurality of first hollow glass bubbles in the liquid; and a plurality of first detector compound molecules not covalently bonded to the plurality of first hollow glass bubbles, the first detector compound molecules comprising a first detectable group that is detected at a first wavelength; wherein the first hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached first bound antibodies (as represented by FIG. 1 or 2).

Embodiment 62 is the bound antibody composition of embodiment 61, wherein the plurality of first detector compound molecules are complexed with the plurality of first bound antibodies (as represented by FIG. 1).

Embodiment 63 is the bound antibody composition of embodiment 61, wherein the plurality of first detector compound molecules are free detector compound molecules (as represented by FIG. 2).

Embodiment 64 is a method of detecting an analyte in a sample (as represented by FIG. 1), the method comprising:

a) providing a bound antibody composition comprising:
a liquid comprising water;
a plurality of first hollow glass bubbles in the liquid, wherein the first hollow glass bubbles have:
   a density of less than 0.60 gram/mole, or up to 0.40 gram/mole;
   a span of less than 1.0, less than 0.8, or less than 0.7;
   optionally, an average diameter of at least 30 micrometers, or at least 40 micrometers; and
   a plurality of covalently attached first bound antibodies; and a plurality of first detector compound molecules complexed with the plurality of first bound antibodies, the first detector compound molecules comprising a first detectable group that is detected at a first wavelength;
b) adding the sample to the composition, the sample containing first free target analyte molecules;
c) allowing the first free target analyte molecules to complex with the first bound antibodies and displace the first detector compound molecules from the plurality of first bound antibodies;
d) allowing the plurality of first hollow glass bubbles to float to an upper concentrated portion of the liquid; and
e) measuring an amount of the first detector compound molecules in a second portion of the liquid that is below the upper concentrated portion, without removing the plurality of first hollow glass bubbles from the liquid.

Embodiment 65 is a method of detecting an analyte in a sample (as represented by FIG. 2), the method comprising:

a) providing a bound antibody composition comprising:
a liquid comprising water;
a plurality of first hollow glass bubbles in the liquid, wherein the first hollow glass bubbles have:
   a density of less than 0.60 gram/mole, or up to 0.40 gram/mole;
   a span of less than 1.0, less than 0.8, or less than 0.7;
   optionally, an average diameter of at least 30 micrometers, or at least 40 micrometers; and
   a plurality of covalently attached first bound antibodies; and
a plurality of first free detector compounds, the first free detector compounds comprising a first detectable group that is detected at a first wavelength;

b) adding the sample to the composition, the sample containing first free target analyte molecules;

c) allowing the first free target analyte molecules to complex with the first bound antibodies, thereby competing with the first free detector compound molecules;

d) allowing the plurality of first hollow glass bubbles to float to an upper concentrated portion of the liquid; and

e) measuring an amount of the remaining detector compound molecules in a second portion of the liquid that is below the upper concentrated portion, without removing the plurality of first hollow glass bubbles from the liquid.

Embodiment 66 is a bound target composition comprising: a liquid comprising water; a plurality of first hollow glass bubbles in the liquid; and a plurality of first labelled antibodies not covalently bonded to the plurality of first hollow glass bubbles, the first labelled antibodies comprising a first detectable group that is detected at a first wavelength; wherein the first hollow glass bubbles have: a density of less than 0.60 gram/mole, or up to 0.40 gram/mole; a span of less than 1.0, less than 0.8, or less than 0.7; optionally, an average diameter of at least 30 micrometers, or at least 40 micrometers; and a plurality of covalently attached first bound target analytes (as represented by FIGS. 3 and 4).

Embodiment 67 is the bound target composition of embodiment 66, wherein the plurality of first detector compound molecules are complexed with the plurality of first bound target analytes (as represented by FIG. 3).

Embodiment 68 is the bound target composition of embodiment 66, wherein the plurality of first detector compound molecules are free detector compound molecules (as represented by FIG. 4).

Embodiment 69 is a method of detecting an analyte in a sample (as represented by FIG. 3), the method comprising:

a) providing a bound target composition comprising:
a liquid comprising water;
a plurality of first hollow glass bubbles in the liquid, wherein the first hollow glass bubbles have:
   a density of less than 0.60 gram/mole, or up to 0.40 gram/mole;
   a span of less than 1.0, less than 0.8, or less than 0.7;
   optionally, an average diameter of at least 30 micrometers, or at least 40 micrometers; and
   a plurality of covalently attached first bound target analyte molecules; and
a plurality of first labelled antibodies complexed with the plurality of first bound target analyte molecules, the first labelled antibodies comprising a first detectable group that is detected at a first wavelength;

b) adding the sample to the composition, the sample containing first free target analyte molecules;

c) allowing the first free target analyte molecules to complex with the first labelled antibodies and displace the first labelled antibodies from the plurality of first bound target analyte molecules;

d) allowing the plurality of first hollow glass bubbles to float to an upper concentrated portion of the liquid; and

e) measuring an amount of the displaced first labelled antibodies complexed with initially first free target analyte molecules in a second portion of the liquid that is below the upper concentrated portion, without removing the plurality of first hollow glass bubbles from the liquid.

Embodiment 70 is a method of detecting an analyte in a sample (as represented by FIG. 4), the method comprising:

a) providing a bound target composition comprising:
a liquid comprising water;
a plurality of first hollow glass bubbles in the liquid, wherein the first hollow glass bubbles have:
   a density of less than 0.60 gram/mole, or up to 0.40 gram/mole;
   a span of less than 1.0, less than 0.8, or less than 0.7;
   optionally, an average diameter of at least 30 micrometers, or at least 40 micrometers; and
   a plurality of covalently attached first bound target analyte molecules; and
a plurality of first free labelled antibodies, the first free labelled antibodies comprising a first detectable group that is detected at a first wavelength;

b) adding the sample to the composition, the sample containing first free target analyte molecules;

c) allowing the first free target analyte molecules to complex with the first labelled antibodies, thereby competing with the first bound target analyte molecules;

d) allowing the plurality of first hollow glass bubbles to float to an upper concentrated portion of the liquid; and

e) measuring an amount of the remaining first labelled antibodies complexed with initially free target analyte molecules in a second portion of the liquid that is below the upper concentrated portion, without removing the plurality of first hollow glass bubbles from the liquid.

### EXAMPLES

Objects and advantages of this invention are further illustrated by the following examples, but the particular materials and amounts thereof recited in these examples, as well as other conditions and details, should not be construed as limiting. These examples are merely for illustrative purposes only. Unless otherwise stated, all amounts are in weight percent.

Materials XLD3000 silica bubbles and glycidyl-functionalized H20 silica bubbles were obtained from 3M Company, St Paul, Minn.

Sodium sulfate and 0.1N NaOH solution was obtained from Avantor, Radnor, Pa.

Ethanol was obtained from Decon Labs, King of Prussia, Pa. 3-Glycidoxypropyltrimethoxysilane, dry dimethylsulfoxide (DMSO) and ethanolamine were obtained from Alfa Aesar (Haverhill, Mass.).

Bovine serum albumin (BSA), TWEEN 20 nonionic detergent, fumonisin B1, aflatoxin B1 (AFB1), and cortisol were obtained from Sigma Aldrich, St. Louis, Mo.

N-hydroxysuccinimide (NHS) and Ethyl-3-(dimethylaminopropyl)carbodiimide (EDC) were obtained from Thermo Scientific (Rockford, Ill.).

Acetic acid, acetone, chloroform, dimethylformamide, methanol, and triethylamine were obtained from EMD Millipore, Billerica, Mass.

TEXAS RED-X succinimidyl ester, EZ-LINK hydrazide-PEG4-biotin, and QDOT streptavidin sampler kit (Q10151MP) were obtained from Thermo Fisher Scientific, Waltham, Mass.

Rabbit anti-mouse IgG, Cy3 labelled, was obtained from Jackson ImmunoResearch, West Grove, Pa.

Anti-cortisol antibodies (XM-210 and CORT-2) were obtained from Abcam, Cambridge, Mass.

Anti-fumonisin antibodies (C153306) were obtained from Lifespan Biosciences, Seattle, Wash.

Anti-fumonisin antibodies (PY715173) and anti-aflatoxin B1 antibodies (DMAB2948) were obtained from Creative Diagnostics, Shirley, N.Y.

Deionized (DI) water was purified using a MILLI-Q water purification system (EMD Millipore, Burlington, Mass.).

SUPELCLEAN LC-Si SPE cartridges and AMICON Ultra 0.5 mL centrifugal filter units (3000 NMWL) were obtained from Millipore Sigma, St. Louis, Mo.

NANOSEP tubes were obtained from Pall Corporation, Port Wash., N.Y.

Size distribution was measured using a Flex dynamic light scattering system from Microtrac Inc. (Montgomery, Pa.).

LCMS data was recorded on an Agilent 1260 Infinity HPLC system with a 6130 quadrupole LC/MS (Agilent Technologies, Santa Clara, Calif.).

Fluorescence was measured on either a Tecan Infinite 200 plate reader (Tecan Group, Ltd., Switzerland) or a Synergy Neo2 plate reader (BioTek Instruments, Winooski, Vt.).

FITC-cortisol (fluorescein isothiocyanate labelled cortisol) was synthesized according to the procedure in M. Pourfarzaneh et al., Clin. Chem. 26(6), 730-733 (1980).

Anti-AFB1 antibodies were labelled with ALEXA FLUOR 680 according to the instructions provided in the ALEXA FLUOR 680 labelling kit (#A20188) from Thermo Fisher Scientific, Waltham, Mass.

### Example 1: Glass Bubble Fractionation

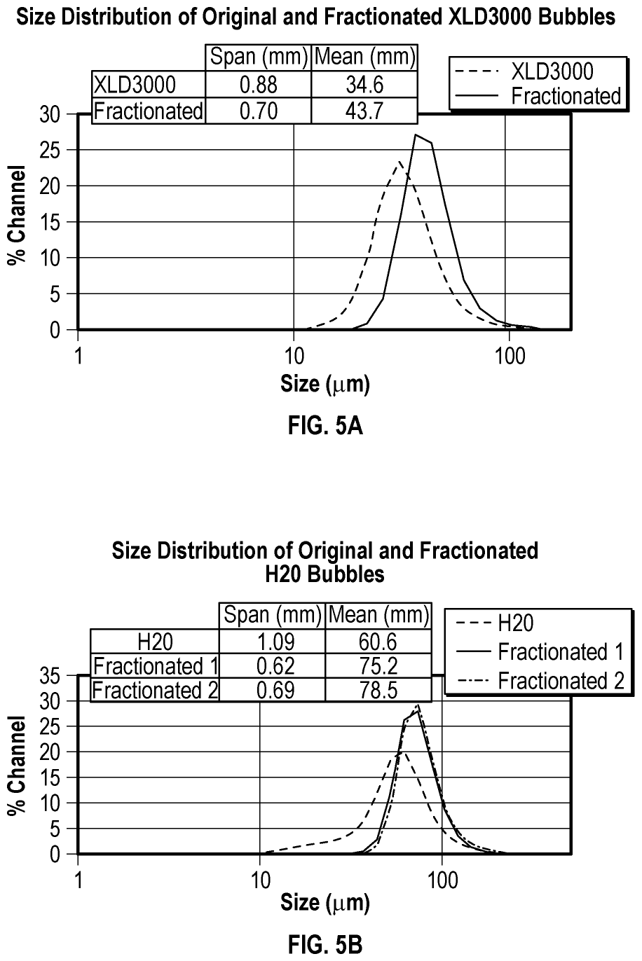

Example 1A—Fractionation of XLD3000 glass bubbles. Glass bubbles XLD3000 (3M ID 98-0212-3469-9; 10 grams (g)) were added to 400 milliliters (mL) of DI water in a separation funnel. The suspension was shaken and the bubbles left to float for 1 minute (min), then 390 mL of liquid (including debris and small glass bubbles) was drained from the bottom. This process was repeated 5 times, after which fractionated glass bubbles were collected by filtering through a Whatman 4 filter under low pressure. They were then rinsed with acetone (100 mL) and left to dry at room temperature overnight. The size distribution (as determined by average (i.e., mean) diameter and span) of the fractionated sample is shown in FIG. 5A.

Example 1B—Fractionation of H20 glass bubbles. Glass bubbles H20 (3M ID 70-0704-8399-8; 10 grams (g)) were added to 400 milliliters (mL) of DI water in a separation funnel. The suspension was shaken and the bubbles left to float for 1 minute (min), then 390 mL of liquid (including debris and small glass bubbles) was drained from the bottom. This process was repeated 5 times, after which fractionated glass bubbles were collected by filtering through a Whatman 4 filter under low pressure. They were then rinsed with acetone (100 mL) and left to dry at room temperature overnight. The size distribution (as determined by average (i.e., mean) diameter and span) of the fractionated sample is shown in FIG. 5B.

### Example 2: Activation and Silanization of XLD3000 Glass Bubbles

Fractionated glass bubbles of Example 1 (6.4 g; mean=43.7 μm; span=0.70 were cleaned by gently rotating in 400 mL of 0.1 Normal (N) NaOH (Thermofisher rotator) at 10 revolutions per minute (rpm) for 3 hours, followed by vacuum filtering with a Whatman #54 filter paper. After rinsing with Milli-Q water, the cleaned glass bubbles were transferred to a plastic dish and dried at room temperature in air overnight.

The cleaned fractionated bubbles (500 milligrams (mg)) were added to 40 mL solution of 95% ethanol/5% 50 millimolar (mM) acetate buffer (pH 5.2) and 300 microliters (μL) of 3-glycidoxypropyltrimethoxysilane, dispersed by vortexing, and then rotated vertically with a rotator at 20 rpm for a period of time between 30 min and 2 hours. The silanized bubbles were then separated from the solution by vacuum filtration (Whatman #54 filter paper), rinsed with ethanol, transferred to a glass vial, and dried in air at room temperature overnight.

### Example 3: Antibody Functionalization of XLD3000 Glass Bubbles

Silanized bubbles of Example 2 (3 mg) were added to a 0.5-mL centrifuge tube and suspended in a solution of 90 μL of coupling buffer (phosphate buffer with 0.9 Molar (M) sodium sulfate, pH 7.5) and 10 μL of 1.5 mg/mL IgG reconstituted stock solution (rabbit anti-mouse IgG antibody, Cyanine3 (Cy3) fluorescent dye labelled). The tube was rotated vertically at 20 rpm for 30 min in the dark, after which the bubbles were vacuum filtered (Whatman #1 filter paper) and rinsed with 1M NaCl solution followed by DI water.

The purpose of this example was to demonstrate efficient binding of antibodies to bubbles. Fluorescence microscopy demonstrated consistent coverage of the bubbles with the antibodies.

### Example 4: General Procedure for Antibody Immobilization on H20 Glass Bubbles Pre-coated with Glycidyl Groups

NANOSEP tubes (0.45 micron) were charged with 10 mg of glycidyl-functionalized H20 silica bubbles. A 300 μL volume of antibody solution (approximately 0.1 mg/mL) in coupling buffer (phosphate buffer with 0.9 M sodium sulfate, pH 7.5) was added and the suspension gently rotated at 25 rpm for 1 hour. Liquid was removed by centrifugation and 0.5 mL of quenching buffer (3 M ethanolamine, pH 9) with 1% BSA was added and mixed by rotation at 25 rpm for 1 hour. Liquid was removed by centrifugation and glass bubbles were washed three times with 0.5 mL of dilution buffer (phosphate buffer, pH 7.4 with 0.10% TWEEN 20 nonionic detergent).

### Example 5: Synthesis of TEXAS RED-X Fumonisin Conjugate (a Detector Compound

A sample of 50 μL of 2 mg/mL fumonisin B1 toxin solution in DMF was mixed with 50 μL of 2 mg/mL TEXAS RED-X succinimidyl ester in methanol (Invitrogen) and 10 μL of triethylamine in a small plastic reaction vial. The reaction mixture was allowed to react overnight at room temperature (approximately 20 hours), protected from light. Samples of 10 μL aliquots of the reaction mixture were loaded onto a SUPELCLEAN LC-Si SPE cartridge and eluted with chloroform/methanol/acetic acid (20:5:0.5). Fractions were collected every 0.5 mL into clean vials and analyzed by LC/MS. Fractions were dried with a gentle stream of nitrogen.

### Example 6: Detection of Cortisol by the Elution Method with Bound XM-210 Antibodies

Anti-cortisol (XM-210 antibodies) functionalized bubbles were prepared according to the general procedure of Example 4. Small quantities of antibody-functionalized bubbles (5 mg) were suspended in 0.5 mL of 100 nM FITC-cortisol (a detector compound) solution in Phosphate Buffered Saline (PBS) and incubated for 1 hour. They were then washed three times with dilution buffer (phosphate buffer, pH 7.4 with 0.1% TWEEN 20 nonionic detergent) to remove excess FITC-cortisol and resuspended in 200 μL of dilution buffer. The bubbles with bound antibodies and complexed detector compound molecules were kept in suspension by vortexing, and 25 μL quantities were carefully aliquoted into separate clean 1.5-mL tubes. Standard solutions of cortisol (1000 parts per billion (ppb), 100 ppb, 10 ppb, 0 ppb, 250 μL of each) were added to the tubes —one standard solution per tube. Each tube was rotated for 30 min, protected from light. The bubbles were allowed to float to the surface while tilting the tube and 160 μL of liquid from each tube was carefully transferred to one well of a black, half area, 96-well plate. Fluorescence intensity (measurement of the emitted light in arbitrary units) was measured at 520 nm emission, with 488 nm excitation.

### Example 7: Detection of Cortisol by the Elution Method with Bound CORT-2 Antibodies

Anti-cortisol (CORT-2 antibodies) functionalized bubbles were prepared according to the general procedure of Example 4. Small quantities of antibody-functionalized bubbles (5 mg) were suspended in 0.5 mL of 100 nM FITC-cortisol (a detector compound) solution in PBS and incubated for 1 hour. They were then washed three times with dilution buffer (phosphate buffer, pH 7.4 with 0.1% TWEEN 20 nonionic detergent) to remove excess FITC-cortisol and resuspended in 200 μL of dilution buffer. The bubbles with bound antibodies and complexed detector compound molecules were kept in suspension by vortexing, and 25 μL quantities were carefully aliquoted into separate clean 1.5-mL tubes. Standard solutions of cortisol (1000 ppb, 100 ppb, 10 ppb, 0 ppb, 250 μL of each) were added to the tubes—one standard solution per tube. Each tube was rotated for 30 min, protected from light. The bubbles were allowed to float to the surface while tilting the tube and 160 μL of liquid from each tube was carefully transferred to one well of a black, half area, 96-well plate. Fluorescence intensity was measured at 520 nm emission, with 488 nm excitation.

Table 1 shows the average fluorescence signal intensity measured from several replicates of solutions containing the indicated amount of cortisol after 30 min incubation with functionalized glass bubbles following the elution method. Two different commercially available anti-cortisol antibodies (CORT-2 and XM210) were used in parallel. Best results were obtained with XM210-functionalized glass bubbles (Example 6) used in elution mode showing a strong correlation between signal intensity and cortisol concentration, as well as clear discrimination between 10 ppb and zero. The CORT-2 antibodies are less effective in this competitive binding assay.

TABLE 1

| Cortisol (ppb) | Example 6 | Example 7 |
|---|---|---|
| 1000 | 2110 | 245 |
| 100 | 1749 | 261 |
| 10 | 918 | 310 |
| 0 | 591 | 211 |

### Example 8: Detection of Cortisol by the Depletion Method with Bound XM-210 Antibodies

Anti-cortisol (XM-210 antibodies) functionalized bubbles were prepared according to the general procedure of Example 4. Small quantities of antibody-functionalized bubbles (5 mg) were then suspended in 300 μL of dilution buffer (phosphate buffer, pH 7.4 with 0.1% TWEEN 20 nonionic detergent) and 50 μL aliquots of this suspension were carefully transferred into clean 1.5-mL tubes containing 50 μL of FITC-cortisol (a detector compound) (100 nM solution in PBS) premixed with 400 μL of cortisol standard solutions (1000 ppb, 100 ppb, 10 ppb, 0 ppb). The samples were incubated with rotation for 30 min. The bubbles were allowed to float to the surface while tilting the tube and 160 μL of liquid from each tube was carefully transferred to one well of a black, half area, 96-well plate. Fluorescence intensity was measured at 520 nm emission, with 488 nm excitation.

### Example 9: Detection of Cortisol by the Depletion Method with Bound CORT-2 Antibodies

Anti-cortisol (CORT-2) functionalized bubbles were prepared according to the general procedure of Example 4. Small quantities of antibody-functionalized bubbles (5 mg) were suspended in 300 μL of dilution buffer (phosphate buffer, pH 7.4 with 0.1% TWEEN 20 nonionic detergent) and 50 μL aliquots of this suspension were carefully aliquoted into clean 1.5-mL tubes containing 50 μL of FITC-cortisol (a detector compound) (100 nM solution in PBS) premixed with 400 μL of cortisol standard solutions (1000 ppb, 100 ppb, 10 ppb, 0 ppb). The samples were incubated with rotation for 30 min. The bubbles were allowed to float to the surface while tilting the tube and 160 μL of liquid from each tube was carefully transferred to one well of a black, half area, 96-well plate. Fluorescence intensity was measured at 520 nm emission, with 488 nm excitation.

Table 2 shows the average fluorescence signal intensity measured from several replicates of solutions containing the indicated amount of cortisol after 30 min incubation with functionalized glass bubbles following the depletion method. Two different commercially available anti-cortisol antibodies (CORT-2 and XM210) were used in parallel. In contrast to the elution method, similar results were obtained with both antibody-functionalized glass bubbles; however, there was reduced sensitivity at the lower concentrations.

TABLE 2

| Cortisol (ppb) | Example 8 | Example 9 |
|---|---|---|
| 1000 | 40791 | 44217 |
| 100 | 39486 | 42460 |
| 10 | 38172 | 41927 |
| 0 | 39697 | 42448 |

### Example 10: Detection of Fumonisin by the Elution Method with Bound Creative Diagnostics

Antibodies

Anti-fumonisin (Creative Diagnostics PY715173) functionalized bubbles were prepared according to the general procedure of Example 4. Small quantities of antibody-functionalized bubbles (5 mg) were suspended in 0.5 mL of 100 nM TEXAS RED-fumonisin conjugate (a detector compound prepared according to Example 5) solution in PBS and incubated for 1 hour. They were then washed three times with dilution buffer (phosphate buffer, pH 7.4 with 0.1% TWEEN 20 nonionic detergent) to remove excess TEXAS RED-fumonisin conjugate and resuspended in 200 μL of dilution buffer. The bubbles with bound antibodies and complexed detector compound molecules were kept in suspension by vortexing, and 25 μL quantities were carefully aliquoted into separate clean 1.5-mL tubes. Standard solutions of fumonisin (100 ppb, 50 ppb, 10 ppb, 0 ppb, 250 μL of each) were added to the tubes—one standard solution per tube. Each tube was rotated for 30 min, protected from light. The bubbles were allowed to float to the surface while tilting the tube and 160 μL of liquid from each tube was carefully transferred to one well of a black, half area, 96-well plate. Fluorescence intensity was measured at 620 nm emission, with 580 nm excitation.

### Example 11: Detection of Fumonisin by the Elution Method with Bound Lifespan Biosciences Antibodies

Anti-fumonisin (Lifespan Biosciences C153306) functionalized bubbles were prepared according to the general procedure of Example 4. Small quantities of antibody-functionalized bubbles (5 mg) were suspended in 0.5 mL of 100 nM TEXAS RED-fumonisin conjugate (a detector compound prepared according to Example 5) solution in PBS and incubated for 1 hour. They were then washed three times with dilution buffer (phosphate buffer, pH 7.4 with 0.1% TWEEN 20 nonionic detergent) to remove excess TEXAS RED-fumonisin conjugate and resuspended in 200 μL of dilution buffer. The bubbles with bound antibodies and complexed detector compound molecules were kept in suspension by vortexing, and 25 μL quantities were carefully aliquoted into separate clean 1.5-mL tubes. Standard solutions of fumonisin (100 ppb, 50 ppb, 10 ppb, 0 ppb, 250 μL of each) were added to the tubes—one standard solution per tube. Each tube was rotated for 30 min, protected from light. The bubbles were allowed to float to the surface while tilting the tube and 160 μL of liquid from each tube was carefully transferred to one well of a black, half area, 96-well plate. Fluorescence intensity was measured at 620 nm emission, with 580 nm excitation.

Table 3 shows the average fluorescence signal intensity measured from several replicates of solutions containing the indicated amount of fumonisin after 30 min incubation with functionalized glass bubbles. Both antibodies tested gave good results, with the signal from solution increasing at higher concentration of target in sample, as well as clear discrimination between 10 ppb and zero.

TABLE 3

| Fumonisin (ppb) | Example 10 | Example 11 |
|---|---|---|
| 100 | 19019.5 | 24226.5 |
| 50 | 18923 | 20091 |
| 10 | 17667 | 16619.5 |
| 0 | 9602 | 8659.5 |

### Example 12: Immobilization of Aflatoxin B1 (Target Analyte) on H20 Glass Bubbles

NANOSEP tubes (0.45 micron) were charged with 10 mg of glycidyl-functionalized H20 silica bubbles. A 400-μL volume of BSA-AFB1 solution (approximately 0.5 mg/mL) in coupling buffer (phosphate buffer with 0.9 M sodium sulfate, pH 7.5) was added and the suspension mixed by rotating at 4° C. overnight. The liquid was then removed by centrifugation and 0.5 mL of quenching buffer (3 M ethanolamine, pH 9) was added and mixed by rotation at 25 rpm for 4 hours. The liquid was removed by centrifugation and the glass bubbles were washed three times with 0.5 mL of dilution buffer (phosphate buffer, pH 7.4 with 0.1% TWEEN 20 nonionic detergent). The bubbles were finally resuspended in dilution buffer and stored at 4° C. until use.

### Example 13: Detection of Aflatoxin by the Bound Target Elution Method

A tube containing 10 mg of aflatoxin B1-coated glass bubbles in dilution buffer (prepared according to Example 12) was centrifuged to remove dilution buffer, washed with dilution buffer (phosphate buffer, pH 7.4 with 0.01% TWEEN 20 nonionic detergent), and centrifuged again. A solution of ALEXA FLUOR 680-labelled antibodies (400 μL of a 10 μg/mL solution in PBS) was added and incubated with rotation for 1.5 hours, protected from light. The bubbles were then washed three times with dilution buffer (phosphate buffer, pH 7.4 with 0.01% TWEEN 20 nonionic detergent) to remove excess antibody, resuspended in dilution buffer (300 μL), and transferred to a 2-mL vial.

The bubbles with bound target analyte molecules and complexed labelled antibodies were kept in suspension by vortexing, and 25 μL quantities were carefully aliquoted into separate clean 1.5-mL tubes. Standard solutions of aflatoxin B1 (50 ppb, 5 ppb, 0.5 ppb, 0 ppb, 500 μL of each) were added to the tubes—one standard solution per tube. Each tube was rotated for 30 min, protected from light. The bubbles were allowed to float to the surface while tilting the tube and 200 μL of liquid from each tube was carefully transferred to a NANOSEP tube and centrifuged to filter out residual bubbles before being transferred to one well of a black, half area, 96-well plate. Fluorescence was measured at 715 nm emission, with 670 nm excitation.

Table 4 shows the average fluorescence signal intensity measured from several replicates of solutions containing the indicated amount of aflatoxin B1 after 30 min incubation with functionalized glass bubbles. The antibodies tested gave good results with the signal from solution increasing at higher concentration of target in sample, as well as clear discrimination between 0.5 ppb and zero.

TABLE 4

| Aflatoxin B1 (ppb) | Example 13 |
|---|---|
| 50 | 19979 |
| 5 | 18923 |
| 0.50 | 13285 |
| 0 | 11870 |

### Example 14: Multiplexed Detection of Fumonisin and Cortisol in the Same Sample Using the Bound Antibody Elution Method

Example 14A—Cortisol bubble preparation. XLD3000 bubbles were first silanized with 3-glycidoxypropyltrimethoxysilane as in Example 2. A NANOSEP tube was then charged with 10 mg of epoxy-functionalized XLD3000 bubbles. A solution of anti-cortisol antibodies (XM-210) was prepared by mixing 25 μL of the 2 mg/mL antibody solution and 375 μL coupling buffer (phosphate buffer with 0.9 M sodium sulfate, pH 7.5), and was then added to the tube containing the bubbles and gently rotated at 25 rpm for 1 hour. The liquid was removed by briefly centrifuging and 0.5 mL of quenching buffer (3 M ethanolamine, pH 9) with 1% BSA was added and the suspension rotated at 25 rpm for 1 hour. The liquid was removed again by brief centrifugation and the glass bubbles were washed three times with 0.5 mL of dilution buffer (phosphate buffer, pH 7.4 with 0.1% TWEEN 20 nonionic detergent). A solution of FITC-cortisol conjugate (500 μL, 100 nM) was added and the suspension was rotated for 1.5 hours, protected from light. The liquid was removed by centrifugation, the bubbles were washed three times with dilution buffer to remove excess conjugate, and then resuspended in 0.25 mL of dilution buffer.

14B—Fumonisin bubble preparation. XLD3000 bubbles were first silanized with 3-glycidoxypropyltrimethoxysilane as in Example 2. A NANOSEP tube was then charged with 10 mg of epoxy-functionalized XLD3000 bubbles. A solution of anti-fumonisin antibodies (Creative Diagnostics PY715173) was prepared by mixing 10 μL of the 5.3 mg/mL antibody solution and 390 μL coupling buffer (phosphate buffer with 0.9 M sodium sulfate, pH 7.5), and was then added to the tube containing the bubbles and gently rotated at 25 rpm for 1 hour. The liquid was removed by briefly centrifuging and 0.5 mL of quenching buffer (3 M ethanolamine, pH 9) with 1% BSA was added and the suspension rotated at 25 rpm for 1 hour. The liquid was removed again by brief centrifugation, and the glass bubbles were washed three times with 0.5 mL of dilution buffer (phosphate buffer, pH 7.4 with 0.1% TWEEN 20 nonionic detergent). A solution of TEXAS RED-X fumonisin conjugate (500 μL, 100 nM, prepared according to Example 5) was added and the suspension was rotated for 1.5 hours, protected from light. The liquid was removed by centrifugation, the bubbles were washed three times with dilution buffer to remove excess conjugate, and then resuspended in 0.25 mL of dilution buffer. 14C—Multiplexed competitive assay by the elution method. Bubble suspensions from 14A and 14B were kept in suspension by vortexing and 25 μL quantities were carefully aliquoted into clean 1.5-mL tubes. Aliquots of target solutions (200 μL each of cortisol alone: 10 ppb, 5 ppb, 1 ppb; fumonisin alone: 10 ppb, 5 ppb, 1 ppb; cortisol+fumonisin: 10 ppb, 5 ppb, 1 ppb of each; control: 0 ppb; n=2 for each sample) were then added to each tube. Each tube was rotated for 1 hour, protected from light. The bubbles were allowed to float to the surface while tilting the tube and 180 μL of liquid from each tube was carefully transferred to one well of a black, half area, 96-well plate. Fluorescence intensity was measured under two sets of conditions: excitation 488 nm/emission 520 nm (cortisol) and excitation 590 nm/emission 620 nm (fumonisin).

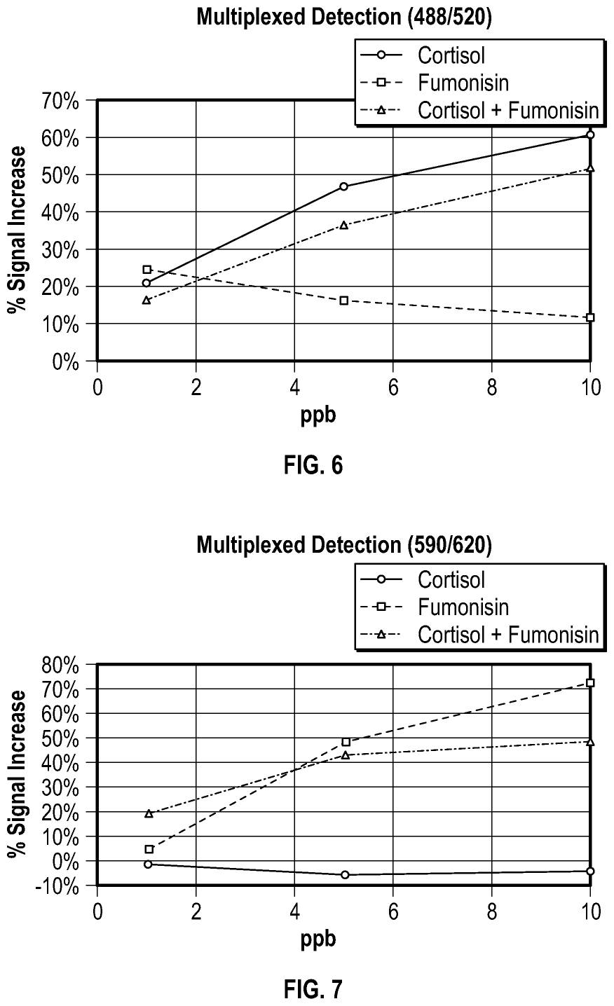

The results are presented in Tables 5 and 6, and FIGS. 6 and 7, as the percentage increase in signal over the control. The data in Table 5 and FIG. 6 clearly demonstrates that a solution containing both cortisol and fumonisin closely tracks the response of a solution containing only cortisol, when interrogated at the appropriate wavelengths for the FITC-cortisol conjugate. The data in Table 6 and FIG. 7 clearly demonstrates that a solution containing both cortisol and fumonisin closely tracks the response of a solution containing only fumonisin, when interrogated at the appropriate wavelengths for the TEXAS RED-X conjugate.

TABLE 5

| | | | Cortisol + |
|---|---|---|---|
| excitation 488/emission 520 | Cortisol | Fumonisin | Fumonisin |
| 10 ppb cortisol, 10 ppb fumonisin, or | 60.5% | 12.0% | 51.9% |
| mixture of 10 ppb of each | | | |
| 5 ppb cortisol, 5 ppb fumonisin, or | 46.9% | 16.3% | 36.6% |
| mixture of 5 ppb of each | | | |
| 1 ppb cortisol, 1 ppb fumonisin, or | 21.1% | 24.3% | 16.5% |
| mixture of 1 ppb of each | | | |

TABLE 6

| | | | Cortisol + |
|---|---|---|---|
| excitation 590/emission 620 | Cortisol | Fumonisin | Fumonisin |
| 10 ppb cortisol, 10 ppb fumonisin, or | −4.1% | 72.8% | 48.6% |
| mixture of 10 ppb of each | | | |
| 5 ppb cortisol, 5 ppb fumonisin, or | −5.8% | 48.6% | 43.3% |
| mixture of 5 ppb of each | | | |
| 1 ppb cortisol, 1 ppb fumonisin, or | −1.3% | 5.1% | 19.7% |
| mixture of 1 ppb of each | | | |

### Example 15: Multiplexed Detection of Aflatoxin, Fumonisin, and Cortisol in the Same Sample Using the Bound Antibody Elution Method

15A—Cortisol bubble preparation. A NANOSEP tube was charged with 12 mg of epoxy-coated H20 glass bubbles. A solution of anti-cortisol antibodies (XM-210) was prepared by mixing 25 μL of the 2 mg/mL antibody solution and 375 μL coupling buffer (phosphate buffer with 0.9 M sodium sulfate, pH 7.5), and was then added to the tube containing the bubbles and gently rotated at 25 rpm for 1 hour. The liquid was removed by briefly centrifuging and 0.5 mL of quenching buffer (3 M ethanolamine, pH 9) with 1% BSA was added and the suspension rotated at 25 rpm for 1 hour. The liquid was removed again by brief centrifugation, and the glass bubbles were washed three times with 0.5 mL of dilution buffer (phosphate buffer, pH 7.4 with 0.10% TWEEN 20 nonionic detergent). A solution of FITC-cortisol conjugate (400 μL, 100 nM) was added and the suspension was rotated for 1.5 hours, protected from light. The liquid was removed by centrifugation, the bubbles were washed three times with dilution buffer, and then resuspended in 0.3 mL of dilution buffer.

15B—Fumonisin bubble preparation. A NANOSEP tube was charged with 12 mg of epoxy-coated H20 glass bubbles. A solution of anti-fumonisin antibodies (Creative Diagnostics PY715173) was prepared by mixing 10 μL of the 5.3 mg/mL antibody solution and 390 μL coupling buffer (phosphate buffer with 0.9 M sodium sulfate, pH 7.5), and was then added to the tube containing the bubbles and gently rotated at 25 rpm for 1 hour. The liquid was removed by briefly centrifuging and 0.5 mL of quenching buffer (3 M ethanolamine, pH 9) with 1% BSA was added and the suspension rotated at 25 rpm for 1 hour. The liquid was removed again by brief centrifugation, and the glass bubbles were washed three times with 0.5 mL of dilution buffer (phosphate buffer, pH 7.4 with 0.1% TWEEN 20 nonionic detergent). A solution of TEXAS RED-X fumonisin conjugate (400 μL, 100 nM, prepared according to Example 5) was added and the suspension was rotated for 1.5 hours, protected from light. The liquid was removed by centrifugation, the bubbles were washed three times with dilution buffer, and then resuspended in 0.3 mL of dilution buffer.

15C—Aflatoxin B1 bubble preparation. A NANOSEP tube was charged with 12 mg of epoxy-coated H20 glass bubbles. BSA-AFB1 solution (0.5 mg/mL in coupling buffer (phosphate buffer with 0.9 M sodium sulfate, pH 7.5)) was added to the tube containing the bubbles and gently rotated at 25 rpm for 1 hour. The liquid was removed by briefly centrifuging and 0.5 mL of quenching buffer (3 M ethanolamine, pH 9, without extra BSA), was added and the suspension rotated at 25 rpm for 1 hour. The liquid was removed again by briefly centrifuging, and the glass bubbles were washed three times with 0.5 mL of dilution buffer (phosphate buffer, pH 7.4 with 0.1% TWEEN 20 nonionic detergent). A solution of ALEXA FLUOR 680-labelled anti-aflatoxin antibody (400 μL, 10 μg/mL) was added and the suspension was rotated for 1.5 hours, protected from light. The liquid was removed by centrifugation, the bubbles were washed three times with dilution buffer, and then resuspended in 0.3 mL of dilution buffer.

15D—Multiplexed competitive assay by the elution method. Bubble suspensions from 15A, 15B, and 15C were combined, allowed to rise, and 0.54 mL of buffer solution were removed from the bottom to leave a glass bubble concentration of 100 mg/mL. The bubbles were kept in suspension by vortexing and 20 μL quantities were carefully aliquoted into clean 2-mL tubes. Aliquots of target solutions (300 μL each of cortisol alone: 50 ppb, 5 ppb; fumonisin alone: 50 ppb, 5 ppb; aflatoxin alone: 50 ppb, 5 ppb; cortisol+fumonisin+aflatoxin B1: 50 ppb, 5 ppb each; control: 0 ppb; n=2 for each sample) were also added. Each tube was rotated for 30 min, protected from light. The bubbles were allowed to float to the surface while tilting the tube, and 130 μL from each duplicate were removed, combined, filtered through a NANOSEP tube, and 170 μL were transferred to one well of a black, half area, 96-well plate. Fluorescence intensity was measured for the filtered samples under three sets of conditions: excitation 488 nm/emission 520 nm (cortisol); excitation 590 nm/emission 620 nm (fumonisin); excitation 670 nm/emission 715 nm (aflatoxin B1).

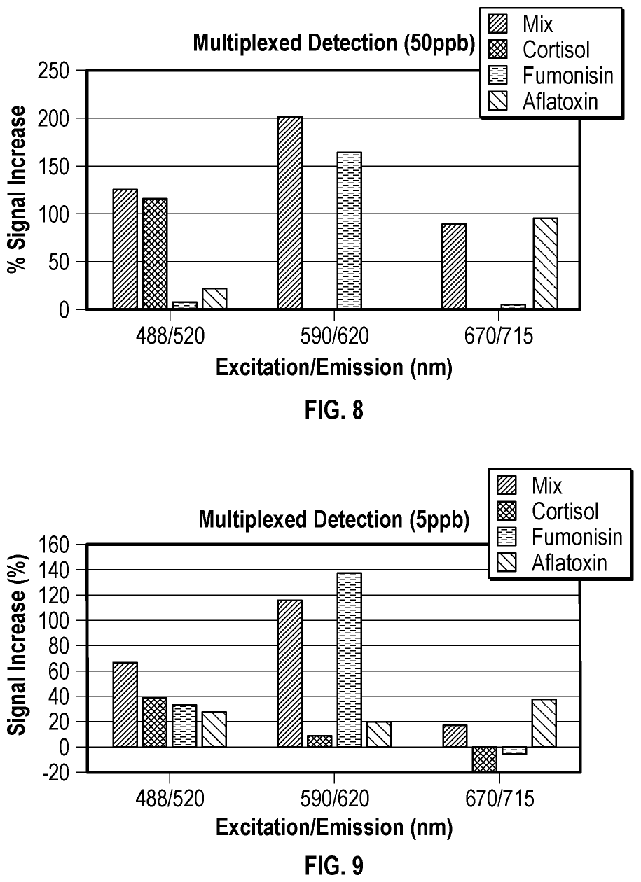

The data is presented in Tables 7 and 8, and FIGS. 8 and 9, as the percentage increase in signal over the control. Table 7 shows the percent increase in fluorescence at different wavelengths for solutions containing 50 ppb of either cortisol, fumonisin, aflatoxin B1, or a mixture containing 50 ppb of each. Table 8 shows the percent increase in fluorescence at different wavelengths for solutions containing 5 ppb of either cortisol, fumonisin, aflatoxin B1, or a mixture containing 5 ppb of each. The data clearly demonstrates that all three targets can be easily detected at 50 ppb, and that fumonisin in particular can be easily detected at 5 ppb, even in the presence of other targets and fluorescent conjugates.

TABLE 7

| wavelength (nm) | Mixture | Cortisol | Fumonisin | Aflatoxin |
|---|---|---|---|---|
| 488/520 | 125 | 116 | 8 | 21 |
| 590/620 | 202 | −9 | 164 | −6 |
| 670/715 | 89 | −18 | 5 | 95 |

TABLE 8

| wavelength (nm) | Mixture | Cortisol | Fumonisin | Aflatoxin |
|---|---|---|---|---|
| 488/520 | 66 | 39 | 33 | 27 |
| 590/620 | 116 | 9 | 137 | 20 |
| 670/715 | 17 | −67 | −6 | 37 |

### Example 16: Detection of Fumonisin in Grain Extracts Using Bound Antibody Elution Method

Samples (2 g) of three different grains (oats, corn, wheat) were weighed into separate 50-mL centrifuge tubes. One of each pair was treated with 10 mL of a 3% solution of TWEEN 20 nonionic detergent in PBS, shaken by hand for three minutes, allowed to settle for 30 seconds, and then the supernatant was decanted, filtered through a 0.45-μm syringe filter, and then diluted 10× with PBS. Sufficient quantities of a 10-ppm standard solution of fumonisin in water were added to either the grain extract or dilution buffer (phosphate buffer, pH 7.4 with 0.10% TWEEN 20 nonionic detergent) to make samples containing 100 ppb, 50 ppb, 5 ppb, and 0 ppb fumonisin.

XLD3000 bubbles were first silanized with 3-glycidoxypropyltrimethoxysilane as in Example 2. Two NANOSEP tubes were then charged with 5 mg of the epoxy-functionalized XLD3000 bubbles each, followed by a solution of anti-fumonisin antibodies (Creative Diagnostics PY715173) prepared by mixing 5 μL of 5.3 mg/mL antibody solution and 395 μL coupling buffer (phosphate buffer with 0.9 M sodium sulfate, pH 7.5). The tubes were gently rotated at 25 rpm for 1 hour, after which the liquid was removed by briefly centrifuging and 0.35 mL of quenching buffer (3 M ethanolamine, pH 9) with 1% BSA was added, and the suspension rotated at 25 rpm for a further hour. The liquid was removed again by brief centrifugation, and the glass bubbles were washed three times with 0.4 mL of dilution buffer (phosphate buffer, pH 7.4 with 0.10% TWEEN 20 nonionic detergent). A solution of TEXAS RED-X fumonisin conjugate (400 μL, 100 nM, prepared according to Example 5) was added and the suspension was rotated for 1 hour, protected from light. The liquid was removed by centrifugation, the bubbles were washed three times with dilution buffer to remove excess conjugate, and then resuspended in 0.2 mL of dilution buffer.

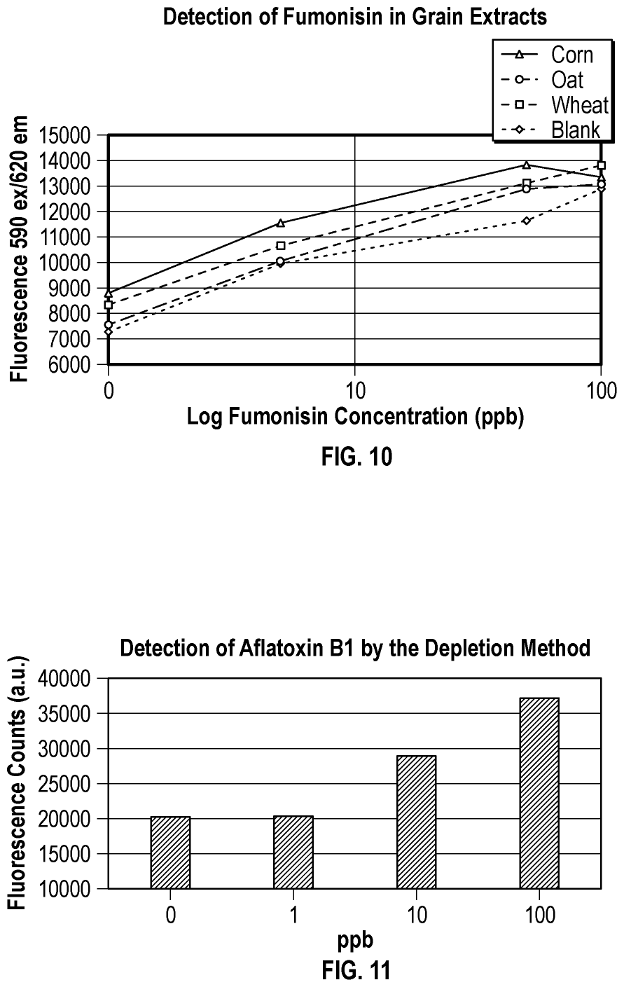

The bubbles were kept in suspension by vortexing and 25 μL quantities were carefully aliquoted into clean 1.5-mL tubes. Target solutions (100 ppb, 50 ppb, 5 ppb and 0 ppb, 250 μL of each in grain extract or PBS control) were added to separate tubes. Each tube was rotated for 1 hour, protected from light. The bubbles were allowed to float to the surface while tilting the tube and 160 μL of liquid from each tube was carefully transferred to one well of a black, half area, 96-well plate. Fluorescence intensity was measured at 620 nm emission, with 590 nm excitation, and is reported in Table 9 and FIG. 10. These data demonstrate that fumonisin can be extracted effectively from grains and analyzed using the methods described herein.

TABLE 9

| | | | | |
|---|---|---|---|---|
| 590 excitation/ | | | | |
| 620 emission | Corn | Oat | Wheat | Control |

| | | | | | |
|---|---|---|---|---|---|
| 100 | ppb | 13351 | 13081 | 13825 | 12893 |
| 50 | ppb | 13822 | 12891 | 13148 | 11648 |
| 5 | ppb | 11548 | 9958 | 10691 | 10025 |
| 0 | ppb | 8796 | 7562 | 8354 | 7315 |

### Example 17: Synthesis of an Aflatoxin B1-QD 585 Conjugate

Dry DMSO (0.4 mL) was added to 50 mg of EZ-LINK hydrazide-PEG4-biotin, to make a 250 mM stock solution. A 10 μL aliquot of this solution (2.5 μmoles) was added to 100 μL of a 10 mg/mL solution of aflatoxin B1 in DMSO in a brown vial. Acetic acid (110 μL) was added to the vial which was then heated to 50° C. overnight in a water bath. LCMS indicated consumption of the EZ-LINK hydrazide-PEG4-biotin and formation of the aflatoxin conjugate (biotin-PEG4-AFB1, MH⁺800.2).

A 5 μL aliquot (2×10-8 mole) of this solution was diluted with 975 μL of phosphate buffer (0.1N pH 7.6) to make a 0.02 nM solution. This solution was added to 20 μL of a 1 μM solution of QDOT 585 streptavidin conjugate (obtained as part of a Q1015IMP sampler kit from Thermo Fisher Scientific) and gently rotated for two hours at room temperature.

After conjugation, the solution was concentrated with two 0.5 mL AMICON Ultra centrifugal filters by centrifuging at 14,000 G for 15 minutes. The eluent (approximately 400 μL) was removed and the concentrated solution left in the filters was washed a further three times on the filter (14,000 G, 15 minutes) with 0.4 mL pH 7.6 buffer. The two concentrated final solutions were combined and refrigerated, yielding approximately 110 μL of a bright orange fluorescent solution containing approximately 10⁻¹¹ moles of QDOT 585, functionalized with aflatoxin B1 through a streptavidin-biotin-PEG4 linker.

### Example 18: Detection of Aflatoxin by the Depletion Method with Bound DMAB2948 Antibodies

Anti-aflatoxin (DMAB2948 antibodies) functionalized bubbles were prepared in a manner analogous to the general procedure of Example 4. Thus, two NANOSEP tubes (0.45 micron) were charged with 10 mg of glycidyl-functionalized XLD3000 silica bubbles. A 400 μL volume of antibody solution (approximately 0.07 mg/mL) in coupling buffer (0.1M phosphate buffer with 0.9 M sodium sulfate, pH 7.5) was added and the suspension gently rotated at 25 rpm for 1 hour.

Liquid was removed by centrifugation and 0.4 mL of quenching buffer (3 M ethanolamine, pH 9) with 1% BSA was added and mixed by rotation at 25 rpm for 1 hour. Liquid was removed by centrifugation and glass bubbles were washed three times with 0.5 mL of dilution buffer (phosphate buffer, pH 7.4 with 0.1% TWEEN 20 nonionic detergent).

The antibody-functionalized bubbles were then suspended in 200 μL of dilution buffer (phosphate buffer, pH 7.4 with 0.10% TWEEN 20 nonionic detergent) and 25 μL aliquots of this suspension were carefully transferred into clean 1.5-mL tubes. A stock solution of aflatoxin B1 (20 μg/mL in methanol, Sigma-Aldrich, St Louis, Mo.) was diluted (PBS, 0.1% Tween-20, pH 7.4) to obtain a 1000 ppb solution. This solution was mixed with QD-PEG-AFB1 conjugate solution from Example 17 (20 uL) and dilution buffer in appropriate quantities to give final aflatoxin B1 concentrations of 100 ppb, 10 ppb, 1 ppb and 0 ppb. Aliquots (175 μL) of these solution were added to the tubes containing antibody-functionalized bubbles in duplicate and incubated with rotation for 30 min. The bubbles were allowed to float to the surface while tilting the tube and 200 μL of liquid from each tube was carefully transferred to one well of a black, half area, 96-well plate. Fluorescence intensity was measured at 585 nm emission, with 400 nm excitation. and is reported in Table 10 and FIG. 11. These data demonstrate that aflatoxin concentrations of 10 ppb and higher can be measured effectively with the depletion method.

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A composition comprising:
a liquid comprising water;
a plurality of first hollow glass bubbles in the liquid, wherein the first hollow glass bubbles have:
a density of less than 0.60 gram/mL;
a span of less than 1.0; and
a plurality of covalently attached first affinity groups; and
a plurality of first detector compound molecules not covalently bonded to the plurality of first hollow glass bubbles, the first detector compound molecules comprising a first detectable group that is detected at a first wavelength.

2. The composition of claim 1, wherein the first affinity groups comprise first bound antibodies.

3. The composition of claim 2, wherein the first bound antibodies are complexed with the first detector compound molecules prior to contact with first free target analyte molecules.

4. The composition of claim 2, wherein the first detector compound molecules comprise a first complementary group and the composition further comprises first free target analyte molecules comprising the first complementary group, wherein the first detector compound molecules compete with the first free target analyte molecules for binding to the first affinity groups.

5. The composition of claim 1, wherein the first affinity groups comprise first bound target analyte molecules and the first detector compound molecules comprise first labelled antibodies.

6. The composition of claim 5, wherein the first affinity groups are complexed with the first labelled antibodies prior to contact with first free target analyte molecules.

7. The composition of claim 5, wherein the composition further comprises first free target analyte molecules, wherein the first bound target analyte molecules compete with the first free target analyte molecules for complexation with the first labelled antibodies.

8. The composition of claim 1, wherein the composition is multiplexed.

9. A method of detecting an analyte in a sample, the method comprising:
a) providing a composition comprising:
a liquid comprising water;
a plurality of first hollow glass bubbles in the liquid, wherein the first hollow glass bubbles have:
a density of less than 0.60 gram/mL;
a span of less than 1.0; and
a plurality of covalently attached first affinity groups; and
a plurality of first detector compound molecules not covalently bonded to the plurality of first hollow glass bubbles, the first detector compound molecules comprising a first detectable group that is detected at a first wavelength;
b) adding the sample to the composition, the sample containing first free target analyte molecules;
c) allowing the first free target analyte molecules to bind to the first affinity groups or the first detector compound molecules;
d) allowing the plurality of first hollow glass bubbles to float to an upper concentrated portion of the liquid; and
e) measuring an amount of the first detector compound molecules in a second portion of the liquid that is below the upper concentrated portion, without removing the plurality of first hollow glass bubbles from the liquid.

10. The method of claim 9, which is a method of detecting a contaminant in a food or water sample.

11. The method of claim 9, wherein measuring the amount of the first detector compound molecules is based on measurement of a fluorescent signal at a first wavelength.

12. The method of claim 9, wherein the first affinity groups comprise first bound antibodies.

13. The method claim 9, wherein the first affinity groups comprise first bound target analyte molecules and the first detector compound molecules comprise first labelled antibodies.

14. The method of claim 9, wherein the composition is multiplexed.

15. The method of claim 14, wherein:
the multiplexed hollow glass bubbles further comprise a plurality of covalently attached second affinity groups different than the first affinity groups; and
the composition further comprises a plurality of second detector compound molecules not covalently bonded to the plurality of multiplexed hollow glass bubbles, the second detector compound molecules comprising a second detectable group that is detected at a second wavelength that is different than the first wavelength;
and/or
the multiplexed composition further comprises:
a plurality of second hollow glass bubbles in the liquid, wherein the second hollow glass bubbles have:
a density of less than 0.60 gram/mL;
a span of less than 1.0; and
a plurality of covalently attached second affinity groups different than the first affinity groups; and
a plurality of second detector compound molecules not covalently bonded to the plurality of second hollow glass bubbles, the second detector compound molecules comprising a second detectable group that is detected at a second wavelength that is different than the first wavelength.
